(19) United States
(12) Patent Application Publication
(10) Pub. No.: US 2016/0237463 A1
(43) Pub. Date: Aug. 18, 2016

(54) METHOD FOR PRODUCING LOWER ALCOHOL FATTY ACID ESTERIFIED PRODUCT-CONTAINING COMPOSITION, AND LOWER ALCOHOL FATTY ACID ESTERIFIED PRODUCT-CONTAINING COMPOSITION

(71) Applicant: KEWPIE CORPORATION, Tokyo (JP)

(72) Inventors: Hideaki Kobayashi, Chofu-shi (JP); Ryosuke Hoshina, Chofu-shi (JP); Minami Kondo, Chofu-shi (JP)

(73) Assignee: KEWPIE CORPORATION, Tokyo (JP)

(21) Appl. No.: 15/025,020

(22) PCT Filed: Sep. 26, 2014
(86) PCT No.: PCT/JP2014/075652

§ 371 Date: Mar. 25, 2016

(30) Foreign Application Priority Data
Sep. 26, 2013 (JP) 2013-200296

(51) Int. Cl. C12P 7/64; A21D 13/08; A21D 2/16

(52) U.S. Cl. CPC C12P 7/6436; A23L 1/3008; A21D 2/16; A21D 13/08; A23V 2002/00

(57) ABSTRACT

Provided is a method of producing a lower alcohol ester of fatty acid-containing composition, the method including treating a raw material oil and fat containing an EPA-containing glyceride with a lipase to obtain a lower alcohol ester of fatty acid-containing composition including a lower alcohol ester of EPA, a content of water in a reaction solution in the treating being 0.4 mass % or more.

### TECHNICAL FIELD

The present invention relates to a method of producing a lower alcohol ester of fatty acid-containing composition and to a lower alcohol ester of fatty acid-containing composition.

### BACKGROUND ART

A polyunsaturated fatty acid and a derivative thereof have many physiological functions, such as reduction of blood lipids, and have been used as a raw material for medicines, cosmetics, foods, or the like for many years. Accordingly, a method of purifying a polyunsaturated fatty acid and a derivative thereof having high purity and good quality has been investigated.

For example, in Patent Literature 1 (JP 59-113099 A), there is a disclosure of a method of producing a lower alcohol ester of fatty acid by treating an oil and fat including fatty acid glycerides under an alkaline condition.

When an oil and fat is treated under an alkaline condition, however, a lower alcohol ester of fatty acid is isomerized at a position of an unsaturated bond to obtain an isomerized compound in some cases. It is often difficult to separate the isomerized compound from a non-isomerized lower alcohol ester of fatty acid. Therefore, a method of efficiently producing a lower alcohol ester of fatty acid with less amount of an isomerized compound is needed.

### CITATION LIST

### Patent Literature

[PTL 1] JP 59-113099 A

### SUMMARY OF INVENTION

### Technical Problem

The present invention provides a method of producing a lower alcohol ester of fatty acid-containing composition by which a lower alcohol ester of fatty acid-containing composition having high purity can be efficiently produced in a simple manner, and a lower alcohol ester of fatty acid-containing composition.

### Solution to Problem

The inventors of the present invention have found that a lower alcohol ester of EPA having high purity is produced by a method involving treating a raw material oil and fat containing an EPA-containing glyceride with a lipase to selectively produce a lower alcohol ester of EPA without treatment under an alkaline condition, and completed the present invention. More specifically, according to the method of producing a lower alcohol ester of fatty acid-containing composition of the present invention, a lower alcohol ester of EPA having less amount of an isomerized compound and high purity can be produced without treatment under an alkaline condition.

1. A method of producing a lower alcohol ester of fatty acid-containing composition according to one embodiment of the present invention includes treating a raw material oil and fat containing an EPA-containing glyceride with a lipase to obtain a lower alcohol ester of fatty acid-containing composition including a lower alcohol ester of EPA, in which a content of water in a reaction solution in the treating is 0.4 mass % or more.

2. In the method of producing a lower alcohol ester of fatty acid-containing composition according to Item 1, the reaction solution in the treating may further include 0.1 part by mass or more and 2.5 parts by mass or less of a lower alcohol relative to 9.5 parts by mass of the raw material oil and fat.

3. In the method of producing a lower alcohol ester of fatty acid-containing composition according to Item 2, the treating may include adding the lower alcohol to the reaction solution in the treating in a continuous or step-wise manner.

4. In the method of producing a lower alcohol ester of fatty acid-containing composition according to any one of Items 1 to 3, the treating may include using an immobilized enzyme obtained by immobilizing the lipase.

5. In the method of producing a lower alcohol ester of fatty acid-containing composition according to any one of Items 1 to 4, in the treating, the immobilized enzyme may have a particulate form.

6. In the method of producing a lower alcohol ester of fatty acid-containing composition according to any one of Items 1 to 5, the lipase may include a 1,3-position-specific lipase.

7. In the method of producing a lower alcohol ester of fatty acid-containing composition according to any one of Items 1 to 6, the reaction solution may have an acid value of 2 or more.

8. In the method of producing a lower alcohol ester of fatty acid-containing composition according to any one of Items 1 to 7, the raw material oil and fat may further contain a DHA-containing glyceride, the lower alcohol ester of fatty acid-containing composition may further include a lower alcohol ester of DHA, and a lower alcohol ester of fatty acid included in the lower alcohol ester of fatty acid-containing composition may have a molar ratio A of the lower alcohol ester of EPA to the lower alcohol ester of DHA (lower alcohol ester of EPA/lower alcohol ester of DHA) higher than a molar ratio B of EPA to DHA (EPA/DHA) in a fatty acid constituting a fatty acid glyceride included in the raw material oil and fat.

9. In the method of producing a lower alcohol ester of fatty acid-containing composition according to Item 8, the molar ratio A and the molar ratio B may have a relationship represented by the following expression (1).

1.5≦A/B (1)

10. In the method of producing a lower alcohol ester of fatty acid-containing composition according to Item 8 or 9, the lower alcohol ester of fatty acid-containing composition may include 40 mass % or more and 90 mass % or less of a lower alcohol ester of fatty acid, and the lower alcohol ester of fatty acid may have a molar ratio of the lower alcohol ester of EPA to the lower alcohol ester of DHA (lower alcohol ester of EPA/lower alcohol ester of DHA) of 3.0 or more and 30 or less.

11. In the method of producing a lower alcohol ester of fatty acid-containing composition according to any one of Items to 10, the lower alcohol ester of fatty acid-containing composition may further include a lower alcohol ester of DHA, and the method may further include distilling the lower alcohol ester of fatty acid-containing composition to separate a mixture of lower alcohol ester of fatty acids including the lower alcohol ester of EPA and the lower alcohol ester of DHA from a component other than the mixture.

12. In the method of producing a lower alcohol ester of fatty acid-containing composition according to Item 11, the mixture may have a ratio of an intensity of a peak which appears around 966 cm⁻¹ to an intensity of a peak which appears around 1,736 cm⁻¹ of 0.075 or less in FT-IR spectral analysis thereof.

13. In the method of producing a lower alcohol ester of fatty acid-containing composition according to Item 11 or 12, the mixture may include 90 mass % or more of a lower alcohol ester of fatty acid, and the lower alcohol ester of fatty acid may include a lower alcohol ester of EPA and a lower alcohol ester of DHA at the following molar ratio:

3.0≦(lower alcohol ester of EPA/lower alcohol ester of DHA)≦30.

14. In the method of producing a lower alcohol ester of fatty acid-containing composition according to any one of Items 11 to 13, the method may further include bringing the mixture into contact with an aqueous silver salt solution.

15. In the method of producing a lower alcohol ester of fatty acid-containing composition according to Item 14, the method may further include distilling the mixture after being brought into contact with the aqueous silver salt solution to separate a lower alcohol ester of fatty acid other than the lower alcohol ester of EPA, to thereby obtain the lower alcohol ester of EPA.

16. In the method of producing a lower alcohol ester of fatty acid-containing composition according to any one of Items 11 to 13, the method may further include distilling the mixture to separate a lower alcohol ester of fatty acid other than the lower alcohol ester of EPA, to thereby obtain the lower alcohol ester of EPA.

17. A lower alcohol ester of fatty acid-containing composition according to one embodiment of the present invention includes 40 mass % or more and 90 mass % or less of a lower alcohol ester of fatty acid, in which the lower alcohol ester of fatty acid includes a lower alcohol ester of EPA and a lower alcohol ester of DHA at the following molar ratio:

3.0≦(lower alcohol ester of EPA/lower alcohol ester of DHA)≦30.

18. The lower alcohol ester of fatty acid-containing composition according to Item 17 may have a ratio of an intensity of a peak which appears around 966 cm⁻¹ to an intensity of a peak which appears around 1,736 cm⁻¹ of 0.15 or less in FT-IR spectral analysis thereof.

19. A lower alcohol ester of fatty acid-containing composition according to one embodiment of the present invention includes 90 mass % or more of a lower alcohol ester of fatty acid, in which the lower alcohol ester of fatty acid includes a lower alcohol ester of EPA and a lower alcohol ester of DHA at the following molar ratio:

3.0≦(lower alcohol ester of EPA/lower alcohol ester of DHA)≦30.

20. The lower alcohol ester of fatty acid-containing composition according to Item 19 may have a ratio of an intensity of a peak which appears around 966 cm⁻¹ to an intensity of a peak which appears around 1,736 cm⁻¹ of 0.075 or less in FT-IR spectral analysis thereof.

21. The lower alcohol ester of fatty acid-containing composition according to any one of Items 17 to 20 may have an acid value of less than 5.

22. A method of producing a lower alcohol ester of fatty acid-containing composition according to one embodiment of the present invention includes distilling the lower alcohol ester of fatty acid-containing composition of Item 17 to separate the lower alcohol ester of fatty acid-containing composition of Item 20 from a component other than the composition.

23. A method of producing a lower alcohol ester of fatty acid-containing composition according to one embodiment of the present invention includes bringing the lower alcohol ester of fatty acid-containing compound of Item 19 or 20 into contact with an aqueous silver salt solution.

24. A method of producing a lower alcohol ester of fatty acid-containing composition according to one embodiment of the present invention includes distilling the lower alcohol ester of fatty acid-containing composition of any one of Items 19 to 21 to separate a lower alcohol ester of fatty acid other than the lower alcohol ester of EPA, to thereby obtain the lower alcohol ester of EPA.

25. A lower alcohol ester of fatty acid-containing composition according to one embodiment of the present invention includes 96.5 mass % or more of a lower alcohol ester of EPA, the lower alcohol ester of fatty acid-containing composition having a ratio of an intensity of a peak which appears around 966 cm⁻¹ to an intensity of a peak which appears around 1,736 cm⁻¹ of 0.085 or less in FT-IR spectral analysis thereof.

26. A method of producing a food composition according to one embodiment of the present invention includes using at least one kind selected from the lower alcohol ester of fatty acid-containing composition obtained by the production method of any one of Items 11 to 16 and 22 to 24, and the lower alcohol ester of fatty acid-containing composition of Item 25 to obtain a food composition.

27. A method of producing a capsule formulation according to one embodiment of the present invention includes using at least one kind selected from the lower alcohol ester of fatty acid-containing composition obtained by the production method of any one of Items 11 to 16 and 22 to 24, and the lower alcohol ester of fatty acid-containing composition of Item 25 to obtain a capsule formulation.

### Advantageous Effects of Invention

The method of producing a lower alcohol ester of fatty acid-containing composition includes treating a raw material oil and fat containing an EPA-containing glyceride with a lipase to obtain a lower alcohol ester of fatty acid-containing composition including a lower alcohol ester of EPA, and when a content of water in the reaction solution in the treating is 0.4 mass % or more, a lower alcohol ester of EPA having high purity can be efficiently produced and the stability of the enzyme (lipase) can be enhanced.

More specifically, the stability of the enzyme is enhanced and hence reuse of the enzyme becomes possible. Accordingly, reduction of the production cost and resource saving can be achieved. In addition, the method can produce a lower alcohol ester of EPA without treating under an alkaline condition, and hence the production of the isomerized compound can be reduced. Through the treatment with a lipase, an EPA-containing glyceride can also be selectively converted to a lower alcohol ester of EPA among a plurality of kinds of fatty acid glycerides.

### BRIEF DESCRIPTION OF DRAWINGS

FIG. 1 is a flow chart of a method of producing a lower alcohol ester of fatty acid-containing composition according to one embodiment of the present invention.

### DESCRIPTION OF EMBODIMENTS

The present invention is hereinafter described in detail with reference to the drawing. In the present invention, “part(s)” means “part(s) by mass” and “%” means “mass o” unless otherwise specified. However, “ratio” and “%” of constituent fatty acids in glycerides, and “ratio” and “%” of fatty acid esters in a lower alcohol ester of fatty acid mean “molar ratio” and “mold” of constituent fatty acids, respectively.

<Method of Producing Lower Alcohol Ester of Fatty Acid-Containing Composition>

A method of producing a lower alcohol ester of fatty acid-containing composition according to one embodiment of the present invention (hereinafter sometimes referred to simply as “production method”) includes treating a raw material oil and fat containing an eicosapentaenoic acid (EPA, C20:5) glyceride with a lipase to obtain a lower alcohol ester of fatty acid-containing composition including a lower alcohol ester of EPA (hereinafter sometimes referred to as “composition according to the embodiment”), a content of water in a reaction solution in the treating being 0.4 mass % or more.

<Lower Alcohol: Definition>

In the present invention, the “lower alcohol” refers to an alcohol having 1, 2, or 3 carbon atoms (methanol, ethanol, n-propyl alcohol, or isopropyl alcohol).

<Lower Alcohol Ester of Fatty Acid: Definition>

In addition, in the present invention, the “lower alcohol ester of fatty acid” refers to a compound obtained by esterifying a carboxyl group (—CO₂H) constituting a fatty acid with a lower alcohol.

<Lower Alcohol Ester of EPA>

In addition, in the present invention, the “lower alcohol ester of EPA” refers to a compound obtained by esterifying a carboxyl group (—CO₂H) constituting eicosapentaenoic acid (EPA) with a lower alcohol. In addition, in the present invention, the “lower alcohol ester of DHA” refers to a compound obtained by esterifying a carboxyl group constituting docosahexaenoic acid (DHA) with a lower alcohol.

<Definition of Glyceride>

In addition, in the present invention, the “glyceride” has a concept of glycerin fatty acid esters including a monoglyceride, a diglyceride, and a triglyceride.

<Definition of EPA-Containing Glyceride>

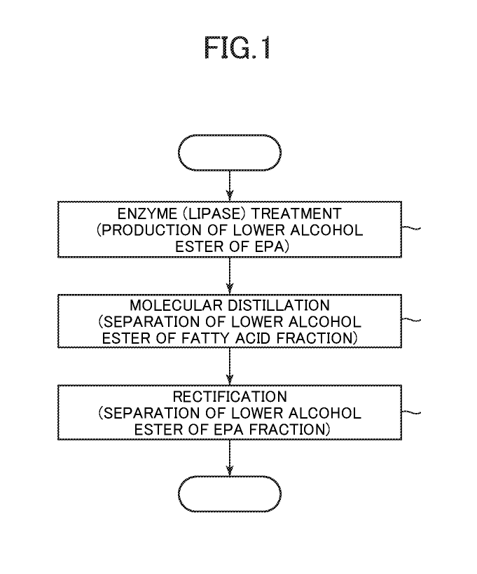

Further, in the present invention, the “EPA-containing glyceride” refers to a compound in which part or all of fatty acid moieties constituting a glycerin fatty acid ester including a monoglyceride, a diglyceride, and a triglyceride are EPA, and has a concept including EPA monoglyceride, EPA diglyceride, and EPA triglyceride. In the present invention, the “DHA-containing glyceride” refers to a compound in which part or all of fatty acid moieties constituting a glycerin fatty acid ester including a monoglyceride, a diglyceride, and a triglyceride are DHA, and has a concept including DHA monoglyceride, DHA diglyceride, and DHA triglyceride.

<Description of FIG. 1>

FIG. 1 is a flow chart of the production method according to the embodiment. In the production method according to the embodiment, first, a raw material oil and fat containing an EPA-containing glyceride is treated with a lipase (Step S1 in FIG. 1) to obtain a composition according to the embodiment including a lower alcohol ester of EPA.

<Enzyme Treatment (Treatment with Lipase)>

In the production method according to the embodiment, a raw material oil and fat including an EPA-containing glyceride is treated with an enzyme (lipase). More specifically, when the raw material oil and fat including the EPA-containing glyceride is brought into contact with a lipase and a lower alcohol so that the lipase acts on the EPA-containing glyceride, the EPA-containing glyceride is selectively converted to a lower alcohol ester of EPA.

<Raw Material Oil and Fat>

The raw material oil and fat used in the production method according to the embodiment only needs to be an oil and fat including a glycerin fatty acid ester containing EPA as a constituent fatty acid (EPA-containing glyceride), and an oil and fat having a content of EPA of 12 mass % or more (generally 20 mass % or less) in a fatty acid composition is preferred. The raw material oil and fat may contain a glyceride containing a fatty acid other than EPA, such as DHA (C22:6), as a constituent fatty acid. When the raw material oil and fat contains a glyceride containing DHA as a constituent fatty acid, an oil and fat having a content of DHA of 15 mass % or less in the fatty acid composition is preferred. In addition, a fatty acid triglyceride other than EPA included in the raw material oil and fat may be a triglyceride of a polyunsaturated fatty acid. The “polyunsaturated fatty acid” refers to an unsaturated fatty acid having 16 or more carbon atoms and two or more double bonds in a molecule, and examples thereof include the above-mentioned EPA and DHA, and arachidonic acid (C20:4), docosapentaenoic acid (C22:5), stearidonic acid (C18:4), linolenic acid (C18:3), and linoleic acid (C18:2).

<Oil and Fat>

The “oil and fat” generally means a triglyceride, but in the pre sent invention, the oil and fat may include other glycerides on which an enzyme (lipase) acts, such as a diglyceride and a monoglyceride.

<Raw Material Oil and Fat>

Examples of the raw material oil and fat include a fish oil, an animal oil other than the fish oil, a vegetable oil, an oil produced by algae or microorganisms, a mixed oil and fat thereof, and a waste oil thereof.

Examples of the fish oil include sardine oil (8 mol % or more and 20 mol % or less of EPA), tuna oil (3 mol % or more and 10 mol % or less of EPA), bonito oil (5 mol % or more and 10 mol % or less of EPA), cod-liver oil (5 mol % or more and 15 mol % or less of EPA), salmon oil (5 mol % or more and 15 mol % or less of EPA), cuttlefish oil (10 mol % or more and 18 mol % or less of EPA), and menhaden oil (5 mol % or more and 15 mol % or less of EPA).

Herein, the content of EPA in each fish oil refers to a proportion of EPA included as a fatty acid constituting a glyceride in the fish oil. The vegetable oil generally contains no EPA or DHA, but for example, soy oil, rapeseed oil, palm oil, or olive oil, which contain containing EPA or DHA, obtained by a gene recombination technology may be used as the raw material oil and fat. Examples of the oil derived from algae or microorganisms include: arachidonic acid-containing oils derived from Mortierella alpina and Euglena gracilis; EPA-containing oils derived from kurome, sea oak, sea mustard, hijiki, Petalonia binghamiae, and Fucus species; and DHA-containing oils derived from Crypthecodinium cohnii, Vibrio marinus, Thraustochytrium aureum, and bacteria belonging to the genus Shewanella. Examples of the animal oil other than the fish oil include whale oil, mutton tallow, beef tallow, lard, butterfat, and egg-yolk oil. In addition, an acid value of the raw material oil and fat is generally 0 or more and 2.5 or less and may also be 0 or more and 2 or less.

The raw material oil and fat may be a raw material oil and fat containing water. In addition, in the present invention, the “waste oil” refers to a fish oil, a vegetable oil and fat, or an animal oil and fat, which has been used, and the waste oil may contain water. When the raw material oil and fat contains both of EPA and DHA, the molar ratio of EPA to DHA in a fatty acid constituting a fatty acid glyceride included in the raw material oil and fat is preferably EPA/DHA=0.5 or more and 6 or less, more preferably 1 or more and 3 or less from the viewpoint that the proportion of the lower alcohol ester of EPA to be obtained can be further increased.

<Enzyme>

An enzyme to be used in the production method according to the embodiment may have any property of crude, partially purified, and purified ones. In addition, the enzyme may be in a free form or immobilized, and an immobilized enzyme in which the lipase is immobilized is preferred from the viewpoint that the enzyme is reusable and post-treatment after the enzyme treatment is easy.

An immobilized enzyme may be immobilized on a carrier. When a content of water in the reaction solution in the enzyme treatment is 0.4 mass % or more, the stability of the enzyme is enhanced. As a result, reuse of the enzyme becomes possible, and hence when an immobilized enzyme in which the lipase is immobilized is used, the immobilized enzyme can be removed from the reaction solution, and as needed, washed with water or the like. The resultant enzyme can be used again. Therefore, the method is excellent in reusability, handling property, and economic efficiency.

<Carrier>

Examples of the carrier include an organic carrier, an inorganic carrier, and an organic-inorganic composite carrier, such as an ion exchange resin, a porous resin, ceramics, calcium carbonate, Celite, a glass bead, activated carbon. In consideration of durability, affinity with a lipase, and the like, it is preferred that the carrier be an ion exchange resin, a porous resin, or ceramics. Examples of the method for immobilization include an inclusion method, a crosslinking method, a physical adsorption method, an ion adsorption method, a covalent binding method, and a hydrophobic binding method. Of those, from the viewpoint of high binding strength, an inclusion method, a crosslinking method, or a covalent binding method is preferred. From the viewpoints of having a large area of contact with a raw material oil and fat and being uniformly dispersed in the raw material oil and fat, the immobilized enzyme preferably has a particulate form. Alternatively, the immobilized enzyme may be immobilized on a film or a membrane.

When the immobilized enzyme has a particulate form, the enzyme maybe immobilized on a particulate carrier. In this case, a particle size of the carrier is preferably 0.01 mm or more and 3 mm or less, more preferably 0.05 mm or more and 1.5 mm or less. In addition, from the viewpoint of excellent dispersibility in the raw material oil and fat, a lower alcohol, and water, a specific gravity of the immobilized enzyme is preferably 0.2 or more and 2.5 or less.

<Lipase>

From the viewpoint of having a transesterification-catalyzing action, the enzyme is preferably, for example, a lipase. The lipase is scientifically specified by indicating the lipase as “lipase (E.C.3.1.1.3)” according to the international enzyme classification.

<Kinds of Lipase>

A lipase to be used in the production method according to the embodiment may be 1,3-position-specific or non-specific. From the viewpoint that, in the composition according to the embodiment, the molar ratio of the lower alcohol EPA ester can be increased, the lipase is preferably a 1,3-position-specific lipase, that is, an enzyme specifically acting on 1,3-positions alone of a triacylglycerol or more preferentially acting on 1,3-positions than 2-position of the triacylglycerol.

<Specific Examples of Lipase>

Examples of the lipase include lipases derived from: filamentous bacteria belonging to the genera Rhizomucor (Rhizomucor miehei), Mucor (Mucor miehei and Mucor java nicus), Aspergillus (Aspergillus oryzae and Aspergillus niger), Rhizopus (Rhizopus sp.), Penicillium (Penicillium roqueforti and Penicillium camembertii), and Thermomyces (Thermomyces lanuginose); yeasts belonging to the genera Candida (Candida antarctica, Candida rugosa, and Candida cylinciracea), and Pichia; bacteria belonging to the genera Pseudomonas (Pseudomonas sp.), Achromobacter (Achromobacter sp.), Burkholderia (Burkholderia sp.), Alcaligenes (Alcaligenes sp.), and Pseudozyma (Pseudozyma sp.) ; and animals, such as pig pancreas. A commercially available lipase may also be used. Examples of the commercially available lipase include a lipase from Rhizopus delemar (Talipase: manufactured by Tanabe Seiyaku Co., Ltd.), a lipase from Candida cylindacea (Lipase OF: manufactured by Meito Sangyo Co., Ltd.), and a lipase belonging to the genus Psendomans (Lipase PS and Lipase AK: manufactured by Amano Pharmaceutical Co., Ltd.). Examples of the immobilized enzyme include a lipase from Rhizomucor miehei (Lipozyme IM60: manufactured by Novo Nordisk; and Lipozyme RMIM: manufactured by Novo Nordisk) and a lipase from Candia antarctica (Novozym 435: manufactured by Novo Nordisk).

<Amount of Enzyme to be Used>

An amount of the enzyme to be used for a reaction is not particularly defined because the amount is determined depending on a reaction temperature, a reaction time, and the like, but in the case of a free form enzyme, the enzyme may be generally added at 1 unit (U) or more and 10,000 U, preferably 5 U or more and 1,000 U per g of a reaction solution, and the amount may be appropriately set. “1 U of enzyme activity” used herein refers to an amount of an enzyme which releases 1 μmol of a fatty acid in 1 minute in hydrolysis of olive oil in the case of a lipase. When the immobilized enzyme is used, the immobilized enzyme may be added so that the amount of the enzyme is 0.1 mass % or more and 200 mass % or less, preferably 1 mass % or more and 20 mass % or less (mass including mass of a carrier) relative to the mass of the reaction solution.

<Reaction Condition>

In the production method according to the embodiment, the reaction solution in the enzyme treatment includes a raw material oil and fat and an enzyme (immobilized enzyme when the immobilized enzyme is used as the enzyme). When a content of water in the reaction solution in the enzyme treatment is 0.4 mass % or more, the stability of the enzyme is enhanced. As a result, reuse of the enzyme becomes possible, and glycerin, which is produced by an enzyme reaction, is introduced in water to prevent the glycerin from solidifying in oil, and hence the enzyme reaction can smoothly proceed. A content of water in the reaction solution in the enzyme treatment is preferably 0.5 mass % or more, more preferably 1 mass % or more, still more preferably 2 mass % or more, and is also preferably 80 mass % or less, more preferably 50 mass % or less. The content may be, for example, 0.4 mass % or more and 10 mass % or less.

<Content of Water>

When a content of water in the reaction solution in the enzyme treatment is less than 0.4 mass %, a catalyst action of the enzyme is inhibited by the glycerin and the like, produced by the reaction in some cases, and the stability of the enzyme decreases in some cases. Meanwhile, when the content is more than 80 mass %, the contact of the EPA-containing triglyceride and the enzyme decreases, and hence a content of the lower alcohol ester of fatty acid to be obtained in a lower alcohol ester of fatty acid-containing composition is reduced in some cases. Water may be added in a sequential manner, may be added in a continuous manner, or may be added in one portion to the reaction solution.

<Lower Alcohol>

The reaction solution in the enzyme treatment may further include 0.1 part by mass or more and 2.5 parts by mass or less of a lower alcohol relative to 9.5 parts by mass of the raw material oil and fat from the viewpoints that a fatty acid can be esterified to obtain an ester and the lower alcohol is miscible with both of water and an oil and fat so that an enzyme reaction can smoothly proceed.

In the enzyme treatment of the raw material oil and fat in the production method according to the embodiment, the lower alcohol (alcohol having 1, 2, or 3 carbon atoms) may be utilized as an ester moiety of the lower alcohol ester of EPA.

From the viewpoint that increasing a yield of a lower alcohol ester of EPA can be increased, a content of the lower alcohol in the reaction solution in the enzyme treatment is preferably 0.1 part by mass or more (preferably 0.3 part by mass or more) relative to 9.5 parts by mass of the raw material oil and fat. Meanwhile, when a content of the lower alcohol in the reaction solution in the enzyme treatment increases, the enzyme is liable to be deactivated. Thus, from the viewpoint that an activity of the enzyme can be maintained, a content of the lower alcohol in the reaction solution in the enzyme treatment is preferably 2.5 parts by mass or less (preferably 2 parts by mass) relative to 9.5 parts by mass of the raw material oil and fat.

In the enzyme treatment, the lower alcohol may be added in a continuous manner, may be added in a step-wise manner, or may be added in one portion to the reaction solution. In the present invention, the phrase “lower alcohol is added in a continuous manner” refers to sequential addition of a lower alcohol, and the phrase “lower alcohol is added in a step-wise manner” refers to a plurality of times of addition of a lower alcohol in a non-sequential manner.

In addition, when the lower alcohol is added in a step-wise manner to the reaction solution, a single amount of the lower alcohol to be added is preferably 0.1 part by mass or more (more preferably 0.3 part by mass or more), and also preferably 2.5 parts by mass or less (more preferably 1 part by mass or less) relative to 9.5 parts by mass of the raw material oil and fat. When the lower alcohol is added in a step-wise or continuous manner to the reaction solution, the total amount of the lower alcohol to be added is preferably 0.1 part by mass or more (more preferably 0.3 part by mass or more) and also preferably 2.5 parts by mass or less (more preferably 2 parts by mass or less) relative to 9.5 parts by mass of the raw material oil and fat.

<Specific Examples of Lower Alcohol>

A lower alcohol is preferably methanol and/or ethanol, more preferably ethanol from the viewpoint of excellent miscibility with water.

<Reaction Temperature and Reaction Time>

The enzyme treatment may be performed at a temperature of the reaction solution of generally more than 25° C. and 80° C. or less (preferably 28° C. or more, more preferably 30° C. or more, and also preferably 50° C. or less, more preferably 45° C. or less). A temperature of the reaction solution in the enzyme treatment may be determined depending on the kind of an enzyme to be used. In addition, a reaction time is generally 2 hours or more and 48 hours or less, preferably 4 hours or more and 36 hours or less.

<Acid Value>

In the enzyme treatment, from the viewpoint that stability of an enzyme can be further enhanced, an acid value of the reaction solution in the enzyme treatment is preferably 2 or more, more preferably 2.2 or more and 12 or less.

In the present invention, the acid value of the reaction solution is a value measured and calculated by the method described below. An acid value of the reaction solution in the enzyme treatment of 2 or more (preferably 2.2 or more and 12 or less) means that the concentration of a free fatty acid in the reaction solution is high. That is, when a concentration of a free fatty acid in the reaction solution is increased, as described in the section <Reaction Pathway> below, a rate of binding of a free fatty acid and a lipase in the reaction solution is increased and hence the stability of the lipase (enzyme) can be enhanced. In the present invention, the “free fatty acid” refers to a fatty acid (non-ester-linked fatty acid) which is not present as a fatty acid ester.

<Measurement Method for Acid Value>

An acid value may be measured according to Standard methods for the analysis of fats, oils and related materials (committee of standard methods of Japan Oil Chemists' Society, eds.: established by Japan Oil Chemists' Society, 2013 version, 1.5 Acid Value of Extract Oil).

Specifically, first, a sample (reaction solution after completion of a reaction) was accurately measured into an Erlenmeyer flask based on a collection amount corresponding to the estimated acid value, and 100 mL of a mixed solvent of ethanol/diethyl ether=1/1 (w/w) was added thereto to completely dissolve the sample. Then, the mixed solution was titrated with a 0.1 mol/L potassium hydroxide ethanolic solution and the titration was terminated at the point when a phenolphthalein solution added to the mixed solution as an indicator continuously colored for 30 seconds or more. The acid value was calculated according to the following equation (3). Acid value=5.611×A×F/B . . . (3)

(In the equation, A represents an amount of a 0.1 mol/L potassium hydroxide ethanolic solution to be used (mL), B represents an amount of the collected sample (g), and F represents a factor of a potassium hydroxide ethanolic solution.)

<Reaction Pathway>

In transesterification of an alcohol with a fatty acid ester, the alcohol is generally subjected to a reaction with the fatty acid ester under the condition of less amount of water (for example, a content of water of 0.1%, disclosed in Example in Patent Literature 1). As one of the reasons, there is given that an ester bond of the fatty acid ester is prevented from being hydrolyzed with water.

On the other hand, the production method according to the embodiment includes treating a raw material oil and fat containing an EPA-containing glyceride with an enzyme (treatment with a lipase) to obtain a lower alcohol ester of fatty acid-containing composition including a lower alcohol ester of EPA, and when a content of water in the reaction solution in the enzyme treatment is 0.4 mass % or more, the stability of the enzyme can be enhanced.

More specifically, when a content of water in the reaction solution in the enzyme treatment is 0.4 mass % or more, the raw material oil and fat is hydrolyzed with water included in the reaction solution to obtain a free fatty acid. As a result, a concentration of the free fatty acid in the reaction solution is increased (an acid value of the reaction solution is increased), and hence the stability of the enzyme is enhanced.

The enhancement of the stability of the enzyme by the increase in concentration of the free fatty acid in the reaction solution (that is, the increase in acid value of the reaction solution) is presumably based on the following mechanism. A free fatty acid is generally easy to bind to a protein because a hydrophobic of an alkyl chain moiety and a hydrophilic site of a carboxyl group moiety in the free fatty acid are easy to interact with a hydrophobic site and a hydrophilic site in a protein molecule. Thus, it is presumed that when a free fatty acid is present in the reaction solution, the free fatty acid binds to a lipase, which is a protein, so that deactivation of the lipase caused by direct contact of a substance other than a reaction substrate in the reaction solution (for example, a lower alcohol in the reaction solution) with the lipase can be prevented, and hence the stability of the lipase (enzyme) can be enhanced.

On the other hand, when a content of water in the reaction solution in the enzyme treatment is less than 0.4 mass %, it is difficult to maintain the stability of the lipase (enzyme). As one of the reasons, there is given that when a content of water in the reaction solution is as low as less than 0.4 mass %, hydrolysis of a raw material oil and fat hardly proceeds, and hence a free fatty acid is hardly produced. Thus, it is presumed that a free fatty acid does not easily to bind to a protein, and hence the above-mentioned deactivation of a protein (lipase) due to the free fatty acid is not prevented, to thereby make it difficult to enhance the stability of the lipase.

<Reaction Product>

The production method according to the embodiment can produce a composition containing a lower alcohol ester of fatty acid, and specifically, can efficiently produce a lower alcohol ester of EPA, by treating a raw material oil and fat containing an EPA-containing glyceride with a lipase. That is, when the raw material oil and fat containing the EPA-containing glyceride is treated with a lipase, the EPA-containing glyceride can be efficiently converted to a lower alcohol ester of EPA. The lower alcohol ester of fatty acid to be obtained may be an ester of a polyunsaturated fatty acid including a lower alcohol ester of EPA.

For example, when the raw material oil and fat includes both of an EPA-containing glyceride and a DHA-containing glyceride, in a lower alcohol ester of fatty acid included in the composition according to the embodiment, which is finally obtained by the treatment with a lipase described above, a molar ratio A of a lower alcohol ester of EPA to a lower alcohol ester of DHA (lower alcohol ester of EPA/lower alcohol ester of DHA) can be higher than a molar ratio B of EPA to DHA (EPA/DHA) in a fatty acid constituting a fatty acid glyceride included in a raw material oil and fat.

In this case, the molar ratio A and the molar ratio B preferably have a relationship represented by the following expression (1), and more preferably have a relationship represented by the expression (2).

1.5≦A/B (1)

2.0≦A/B≦25 (2)

<Content of Lower Alcohol Ester of Fatty Acid and Lower Alcohol Ester of EPA:Lower Alcohol Ester of DHA>

In addition, the lower alcohol ester of fatty acid-containing composition to be finally obtained includes 40 mass % or more and 90 mass % or less of a lower alcohol ester of fatty acid, and in a lower alcohol ester of fatty acid included in the lower alcohol ester of fatty acid-containing composition, a molar ratio of the lower alcohol ester of EPA to the lower alcohol ester of DHA (lower alcohol ester of EPA:lower alcohol ester of DHA) may be 3.0 or more and 30 or less, and more specifically, the ratio is preferably 3.0 or more and 20 or less, more preferably 3.0 or more and 15 or less.

More specifically, a lower alcohol ester of fatty acid-containing composition obtained by the enzyme treatment includes 40 mass % or more and 90 mass % or less (more specifically, 50 mass % or more and 80 mass % or less) of a lower alcohol ester of fatty acid, and the lower alcohol ester of fatty acid may include a lower alcohol ester of EPA and a lower alcohol ester of DHA at a molar ratio of 3.0≦(lower alcohol ester of EPA/lower alcohol ester of DHA)≦30 (preferably 3.0≦(lower alcohol ester of EPA/lower alcohol ester of DHA)≦20, more preferably 3.0≦(lower alcohol ester of EPA/lower alcohol ester of DHA)≦15).

<Molecular Distillation>

Then, the production method according to the embodiment may further include distilling the lower alcohol ester of fatty acid-containing composition including a lower alcohol ester of EPA obtained by enzyme treatment (treatment with a lipase) (Step S1 in FIG. 1) (i.e., molecular distillation (primary distillation), Step S2 in FIG. 1).

Molecular distillation is distillation performed under a high degree of vacuum. Through the molecular distillation step, a mixture of the lower alcohol ester of fatty acids including a lower alcohol ester of EPA and a lower alcohol ester of DHA can be separated from components other than the mixture (fatty acid monoglyceride, fatty acid diglyceride, and fatty acid triglyceride other than the lower alcohol ester of fatty acid). The fatty acid monoglyceride, fatty acid diglyceride, and fatty acid triglyceride other than the mixture can be utilized for esterification in the presence of a lower alcohol again to provide a lower alcohol ester of DHA.

The molecular distillation may be generally performed under a higher degree of vacuum than that in rectification described below.

The molecular distillation is performed at, for example, a temperature of 80° C. or more and 200° C. or less (preferably 150° C. or more and 200° C. or less) and a degree of vacuum of 0.001 Torr or more and 5 Torr or less (preferably 0.01 Torr or more and 1 Torr or less), and more specifically, a temperature of 140° C. or more and 160° C. or less and a degree of vacuum of 0.01 Torr or more and 0.1 Torr or less.

The molecular distillation is generally performed using an apparatus which can separate the mixture of the lower alcohol ester of fatty acids including the lower alcohol ester of EPA and the lower alcohol ester of DHA from components other than the mixture, and more specifically, the distillation may be generally performed using a commercially available molecular distillation apparatus, for example, a centrifugal molecular distillation apparatus, a short-path distillation apparatus, a falling-film distillation apparatus, or the like. In particular, a short-path distillation apparatus is preferred. A molecular distillation apparatus can separate a low molecular component to be liquefied from a high molecular component not to be liquefied by volatizing a substance to be treated through an evaporator and then passing the volatilized substance through a condenser.

In addition, the molecular distillation is preferably performed before rectification described below, and further, the silver treatment described below is preferably performed after the molecular distillation.

<Peak Intensity Ratio>

In FT-IR spectral analysis of the mixture, a ratio of the intensity of a peak which appears around 966 cm⁻¹ to the intensity of a peak which appears around 1,736 cm⁻¹ is preferably 0.075 or less, more preferably 0.07 or less.

<Significance of Peak Intensity Ratio>

In FT-IR spectral analysis of the mixture, the peak which appears around 1,736 cm⁻¹ represents an ester bond included in a lower alcohol ester of fatty acid. In addition, the peak which appears around 966 cm⁻¹ represents an isomerized compound (isomerized compound including a trans double bond) of a lower alcohol ester of fatty acid included in a lower alcohol ester of fatty acid (quantitation of a trans fatty acid by FTIR, SHIMAZU APPLICATION NEWS No. 430A, Shimadzu Corporation).

Thus, in FT-IR spectral analysis of the mixture, a higher ratio of the intensity of the peak which appears around 966 cm⁻¹ to the intensity of the peak which appears around 1,736 cm⁻¹ indicates that a larger amount of an isomerized compound (including a trans double bond caused by isomerization at a position of a cis double bond included in a fatty acid) is included in a lower alcohol ester of fatty acid. Accordingly, in FT-IR spectral analysis of the mixture, a ratio of the intensity of the peak which appears around 966 cm⁻¹ to the intensity of the peak which appears around 1,736 cm⁻¹ of 0.075 or less indicates that less amount of an isomerized compound is included in the mixture.

<Definition of Isomerized Compound>

In the present invention, the “isomerized compound” of a compound refers to a compound which has an equal molecular formula to the compound, but has a different molecular structure from the compound (isomer), and conversion of a compound to an isomer thereof is termed as isomerization.

For example, a fatty acid constituting a natural oil and fat, such as EPA or DHA, has double bonds all of which form cis configurations and have unconjugated structures. Examples of the isomerization of the fatty acid include the conversion of at least part of a double bond of the fatty acid to a trans configuration and the shifting of the double bond into a position at which the double bond is conjugated.

<Composition of Mixture>

In addition, from the viewpoint that the purity of a lower alcohol ester of EPA obtained by rectification described below can be further increased, it is more preferred that the mixture include 90 mass % or more of the lower alcohol ester of fatty acid, and the lower alcohol ester of fatty acid include a lower alcohol ester of EPA and a lower alcohol ester of DHA at the following molar ratio.

3.0≦(lower alcohol ester of EPA/lower alcohol ester of DHA)≦30.

3.0≦(lower alcohol ester of EPA/lower alcohol ester of DHA)≦20 is still more preferred.

According to the production method of the embodiment, through the enzyme treatment, an EPA-containing glyceride can be selectively converted to a lower alcohol ester of fatty acid, and hence through the molecular distillation step, a lower fatty acid ester including the lower alcohol ester of EPA and the lower alcohol ester of DHA can be separated from other components (fatty acid glyceride other than the lower alcohol ester of fatty acid, and glycerin) by general separation treatment with relative ease. Accordingly, through a rectification step described below, a lower alcohol ester of EPA having high purity can be efficiently produced by a simple method.

<Rectification>

Next, the production method according to the embodiment may further include distilling a mixture including the lower alcohol ester of EPA and the lower alcohol ester of DHA obtained by the molecular distillation (Step S2 in FIG. 1) (rectification, Step S3 in FIG. 1) to separate the lower alcohol ester of fatty acid other than the lower alcohol ester of EPA to obtain a lower alcohol ester of EPA (purity of preferably 96.5 mass % or more, more preferably 98 mass % or more and about 100 mass % or less).

Rectification is distillation performed under a lower degree of vacuum as compared to the molecular distillation described above. Specifically, the rectification is a continuous distillation operation in which liquid is countercurrently brought into contact with vapor in a column with appropriate reflux so that evaporation of the liquid and condensation of the vapor are repeatedly performed, resulting in improved separation accuracy, and is most frequently used for separation and purification of a liquid mixture. Through a rectification step, any lower alcohol ester of fatty acid can be separated from a lower alcohol ester of fatty acid including esters of a plurality of kinds of fatty acids. Therefore, in the rectification step in the embodiment, a lower alcohol ester of EPA can be selectively produced from a plurality of kinds of lower alcohol ester of fatty acids obtained by the molecular distillation step.

The rectification may be generally performed under a lower degree of vacuum than that in the molecular distillation described above.

The rectification is performed at, for example, a temperature of 150° C. or more and 250° C. or less (preferably 160° C. or more and 230° C. or less) and a degree of vacuum of 0.01 Torr or more and 10 Torr or less (preferably 0.1 Torr or more and 5 Torr or less), and more specifically, a temperature of 170° C. or more and 220° C. or less and a degree of vacuum of 0.5 Torr or more and 3 Torr or less.

The rectification is generally performed using an apparatus which can separate the lower alcohol ester of fatty acid other than the lower alcohol ester of EPA from the lower alcohol ester of EPA, and more specifically, the rectification may be performed using a distillation apparatus including a rectifier or a falling-film type distillation apparatus. For example, a plate-type, filling-type, or spring-type rectifier may be used as the rectifier, and a plate-type distillation apparatus having a plate structure or a filling-type distillation apparatus is particularly preferably used. In the distillation apparatus having a plate structure, volatilized substances ascend and the substances remain on different plates depending on the kinds of the substances, and hence the apparatus includes plates set so as to transfer the target substance upward into a plate with a discharge port. In addition, a falling-film type distillation apparatus having relatively less thermal hysteresis is preferably adopted as a heating method. In addition, a distillation method for rectification may be performed in a batch-wise or continuous manner, preferably a continuous manner. The number of theoretical plates of the rectifier may be appropriately set to preferably 2 or more, more preferably 5 or more (generally 2 or more and 10 or less).

In addition, the number of times and the order of the rectification are not limited. That is, the rectification may be performed two times or more (generally two times or more and four times or less) or silver treatment may be performed after the rectification, or the rectification may be performed after the silver treatment. Further, the rectification and the silver treatment are performed and then the rectification may be performed again.

<Silver Treatment>

The production method according to the embodiment may further include silver treatment (treatment involving bringing a lower alcohol ester of fatty acid-containing composition according to the embodiment into contact with an aqueous silver salt solution).

In the production method according to the embodiment, the inventors of the present invention have found that when the reaction solution in the enzyme treatment includes water (for example, in the case where a content of water in the reaction solution in the enzyme treatment is 0.4 mass % or more), a free fatty acid tends to be produced in the enzyme treatment.

The production method according to the embodiment can reduce a content of a free fatty acid in the lower alcohol ester of fatty acid-containing composition according to the embodiment by performing the silver treatment after the enzyme treatment.

That is, a content of a free fatty acid can be reduced by the silver treatment, and hence, for example, the acid value of the lower alcohol ester of fatty acid-containing composition according to the embodiment can be less than 5 (preferably less than 4).

From viewpoint that the production method of the embodiment can reduce the free fatty acid produced by the enzyme treatment, the silver treatment is preferably performed after the enzyme treatment, and for example, the silver treatment may be performed for the mixture obtained by the enzyme treatment (for example, first composition), for a lower alcohol ester of fatty acid-containing composition (for example, second composition) obtained after the molecular distillation, or for a lower alcohol ester of fatty acid-containing composition (for example, second composition) obtained after the rectification.

(Concentration of Silver)

Any silver salt may be used in the silver treatment as long as the silver salt can be complexed with an unsaturated bond in an unsaturated fatty acid, and examples thereof include silver nitrate, silver perchlorate, silver acetate, silver trichloroacetate, and silver trifluoroacetate. Such silver salt is dissolved in water to obtain an aqueous silver salt solution having a concentration of preferably 15 mass % or more, more preferably 20 mass % or more, still more preferably 40 mass % or more. In addition, in a concentration of the silver salt in the aqueous silver salt solution, a saturated concentration may be set as the upper limit.

In addition, in the silver treatment, the aqueous silver salt solution may be recovered and brought into contact with an adsorbent before reuse. Examples of the adsorbent include activated carbon, activated alumina, activated clay, acid clay, a silica gel, diatomaceous earth, aluminum oxide, and magnesium oxide, and one or two or more kinds thereof may be used.

A method of bringing the aqueous silver salt solution into contact with the adsorbent, which is not particularly limited, is, for example, a method involving loading and stirring the adsorbent in the aqueous silver salt solution, or a method involving filling a column with the adsorbent and passing the aqueous silver salt solution through the filled column. Alternatively, the aqueous silver salt solution maybe recovered and be subjected to dilution and/or concentration adjustment, or may be subjected to extraction with an organic solvent before reuse. Concentration adjustment of the recovered aqueous silver salt solution may be performed by evaporating water through reduced pressure or heating, or appropriately adding the silver salt or water with measuring its specific gravity.

In addition, in the silver treatment, the aqueous silver salt solution is added to a substance to be subjected to the silver treatment (for example, a mixture obtained by the enzyme treatment (e.g., first composition), or a composition obtained by the molecular distillation (e.g., second composition)) and the mixture is stirred for preferably 5 minutes or more and 4 hours or less, more preferably 10 minutes or more and 2 hours or less to form a water-soluble complex of the silver salt and a free fatty acid. The complex can be selectively dissolved in the aqueous silver salt solution. When the aqueous silver salt solution is removed, the free fatty acid can be removed. With this, an acid value of a lower alcohol ester of fatty acid-containing composition can be set to less than 5 (preferably less than 4).

In addition, as a temperature for a reaction of the substance to be subjected to the silver treatment with the aqueous silver salt solution, its lower limit may be any temperature such that the aqueous silver salt solution is in a liquid, and its upper limit is up to 100° C., but the reaction temperature is preferably 10° C. or more and 30° C. or less in consideration of, for example, the oxidation stability of a lower alcohol ester of fatty acid-containing composition, the solubility of a silver salt in water, and a production speed of the complex.

It is preferred that the contact of the substance to be subjected to the silver treatment with the aqueous silver salt solution be performed under an inert gas, for example, nitrogen atmosphere, with shielding light in consideration of the oxidation stability of a lower alcohol ester of fatty acid-containing composition, and the stability of a silver salt.

In addition, an organic solvent which is sparingly miscible with water may be added to the aqueous silver salt solution after the contact with the substance to be subjected to the silver treatment. After the addition of the organic solvent, the organic phase is recovered, and a lower alcohol ester of fatty acid-containing composition can be recovered from the recovered phase. In this case, a content of the organic solvent which is sparingly miscible with water is preferably 10 mass % or more and 200 mass % or less, more preferably 30 mass % or more and also 150 mass % or less relative to 100 mass % of an aqueous silver salt solution.

Examples of the organic solvent which is sparingly miscible with water include hydrocarbons, such as linear aliphatic hydrocarbons (e.g., linear aliphatic hydrocarbon having 5 or more and 10 or less carbon atoms, such as n-pentane, n-hexane, n-heptane, n-hexene, n-octane, or isooctane), alicyclic hydrocarbons (alicyclic hydrocarbon having 5 or more and 10 or less carbon atoms, such as cyclohexane, cyclohexene, or methylcyclohexene), or aromatic hydrocarbons (e.g., aromatic hydrocarbon having 5 or more and 10 or less carbon atoms, such as toluene, benzene, ethylbenzene, xylene, or styrene), and petroleum ether. The recovery step may be repeatedly performed a plurality of times. <Specific Example of Lower Alcohol Ester of EPA>

A lower alcohol ester of EPA can be used as a raw material for medicines, cosmetics, foods, or the like. Examples of the lower alcohol ester of EPA include EPA methyl ester, EPA ethyl ester, EPA n-propyl ester, and EPA isopropyl ester. Of those, EPA ethyl ester (sometimes referred to as “EPAEE” herein) is used as a therapeutic for circulatory diseases, such as hyperlipidemia and obstructive arteriosclerosis. Thus, the lower alcohol ester of EPA may be EPAEE, and the lower alcohol ester of DHA may be DHA ethyl ester (sometimes referred to as “DHAEE” herein). <Application of Lower Alcohol Ester of EPA>

In addition, the lower alcohol ester of EPA and the lower alcohol ester of DHA can each be used as a raw material for a food composition or a capsule formulation, such as a supplement.

<Action and Effect-Known Method of Producing Lower Alcohol Ester of Fatty Acid-Containing Composition>

Before an action and effect of the production method in the embodiment is described, a known method of producing a lower alcohol ester of fatty acid-containing composition is described.

<Known Method of Producing Lower Alcohol Ester of Fatty Acid-Containing Composition>

In a known method of producing a lower alcohol ester of fatty acid-containing composition (disclosed in Patent Literature 1), first, a raw material oil and fat including a fatty acid glyceride having a plurality of fatty acids in a lower alcohol fatty acid ester moiety is treated with an alcohol under an alkaline condition, i.e., through a transesterification reaction of the fatty acid glyceride with the alcohol, to provide a lower alcohol ester of fatty acid. In this method, a mixture of a plurality of kinds of lower alcohol ester of fatty acids can be obtained.

<Problem of Known Method of Producing Lower Alcohol Ester of Fatty Acid-Containing Composition>

In order to separate a desired lower alcohol fatty acid ester from a mixture of a plurality of kinds of lower alcohol fatty acid esters, it is required, for example, to perform distillation under very strictly controlled conditions (such as degree of vacuum, heating temperature, heating method, and heating time). As described above, when distillation is performed under the strict conditions, heavy burden is imposed on the production process.

For example, when the transesterification reaction of an oil and fat including an EPA-containing glyceride and a DHA-containing glyceride is performed under an alkaline condition, it is required to perform distillation under a very precisely controlled condition in order to separate a lower alcohol ester of EPA from a lower alcohol ester of DHA (for example, EPAEE and DHAEE) to be obtained because EPA and DHA are close to each other in terms of the number of carbon atoms and the number of double bonds, and hence heavy burden is imposed on the production process. In addition, in general, it is difficult to isolate any one of a lower alcohol ester of EPA and a lower alcohol ester of DHA, and hence a loss tends to occur at the time of distillation.

In addition, distillation generally requires a large-scale apparatus in many cases and hence the production cost tends to increase. Further, when treatment is performed under an alkaline condition, an alkaline solution to be used remains as a waste solution and hence a problem arises in that it is required to treat the waste solution.

Further, in the transesterification reaction, under an alkaline condition, isomerization is caused at a double bond included in a lower alcohol ester of fatty acid (for example, production of a trans double bond) in some cases. In general, isomerization is easily caused by alkaline treatment or heating.

In general, it is difficult to separate an isomerized compound of a lower alcohol ester of fatty acid from a lower alcohol ester of fatty acid by general treatment for removing impurities (for example, distillation or chromatography). Therefore, once an isomerized compound of a lower alcohol ester of fatty acid (having a trans double bond) is produced by treatment under an alkaline condition described above (hereinafter sometimes referred to simply as “alkaline treatment”) or heating treatment, it is generally difficult to remove the isomerized compound from the lower alcohol ester of fatty acid. That is, this isomerized compound is a causative substance which decreases the purity of a lower alcohol ester of fatty acid.

### Action and Effect of Production Method According to the Embodiment

(i) In contrast, the production method according to the embodiment includes, first, treating a raw material oil and fat containing an EPA-containing glyceride with an enzyme (treatment with a lipase) to obtain a lower alcohol ester of fatty acid-containing composition including a lower alcohol ester of EPA, and with this, a lower alcohol ester of EPA having high purity can be efficiently produced.

More specifically, the production method according to the embodiment can inhibit production of an isomerized compound because the method produces a lower alcohol ester of EPA without alkaline treatment. Thus, the method can efficiently produce a lower alcohol ester of EPA having high purity. In addition, in the production method according to the embodiment, a problem regarding treatment of a waste solution does not arise unlike the case of using an alkali, resulting in less damage to the environment.

(ii) Secondly, when the enzyme treatment is performed, a molar ratio of a lower alcohol ester of EPA to a lower alcohol ester of DHA can be increased (specifically, 3.0≦(lower alcohol ester of EPA/lower alcohol ester of DHA)≦30, preferably 3.0≦(lower alcohol ester of EPA/lower alcohol ester of DHA)≦20).

More specifically, when the enzyme treatment is performed, a lower alcohol ester of fatty acid-containing composition including a lower alcohol ester of EPA and a lower alcohol ester of DHA at a molar ratio of 3.0≦(lower alcohol ester of EPA/lower alcohol ester of DHA)≦30 (more preferably 3.0≦(lower alcohol ester of EPA/lower alcohol ester of DHA)≦20) can be produced, and DHA which has not been converted to a lower alcohol ester of DHA remains as a DHA-containing glyceride.

A lower alcohol ester of EPA tends to be relatively easily separable from other components (mainly, a DHA-containing glyceride, glycerin, and a free fatty acid) and a lower alcohol fatty acid ester which has a smaller number of carbon atoms than that of EPA by general treatment for removing impurities, such as distillation and chromatography.

Therefore, when a lower alcohol ester of EPA is separated from other components other than a lower alcohol ester of fatty acid and a lower alcohol fatty acid ester having a smaller number of carbon atoms than that of EPA, precisely controlled distillation for separation of a lower alcohol ester of EPA from other lower alcohol ester of fatty acids, which is generally performed after the alkaline treatment described above, need not to be performed and a loss can also be decreased at the time of distillation. As described above, the production method according to the embodiment can efficiently produce a lower alcohol ester of EPA having high purity in a simple manner by increasing a molar ratio of a lower alcohol ester of EPA to a lower alcohol ester of DHA.

In treating a raw material oil and fat with a lipase, it is presumed that a fatty acid glyceride having EPA as a fatty acid ester moiety is preferentially ethyl-esterified as compared to a fatty acid glyceride containing DHA as a fatty acid ester moiety. Therefore, in the subsequent step, a molar ratio of a lower alcohol ester of EPA to a lower alcohol ester of DHA (lower alcohol ester of EPA/lower alcohol ester of DHA) can be increased.

Thus, the production method according to the embodiment includes first treating a raw material oil and fat containing an EPA-containing glyceride with an enzyme to obtain a lower alcohol ester of fatty acid-containing composition having an increased proportion of a lower alcohol ester of EPA, and then subjecting the resultant composition to distillation treatment, and hence the method is useful in that a lower alcohol ester of EPA having a reduced isomerized compound can be easily isolated in a larger amount.

(iii) Thirdly, when a content of water in the enzyme treatment is 0.4 mass % or more, by virtue of the water included in the reaction solution, the glycerin produced by the enzyme reaction is introduced into water to prevent the glycerin from solidifying in oil and hence the enzyme reaction can smoothly proceed. In addition, a raw material oil and fat is hydrolyzed to generate a free fatty acid, and as a result, a concentration of the free fatty acid in the reaction solution is increased (the acid value of the reaction solution is increased), resulting in enhancement of the stability of the enzyme. As a result, reuse of the enzyme becomes possible, and hence reduction of the production cost and resource saving can be achieved. Thus, the enzyme treatment has excellent reusability, handling property, and economic efficiency, and hence the treatment is suitable for large-scale treatment in addition to small-scale treatment.

<First Composition>

A lower alcohol ester of fatty acid-containing composition according to one embodiment of the present invention (hereinafter sometimes referred to as “first composition”) includes 40 mass % or more and 90 mass % or less (more specifically 50 mass % or more and 80 mass % or less) of a lower alcohol ester of fatty acid, and the lower alcohol ester of fatty acid includes a lower alcohol ester of EPA and a lower alcohol ester of DHA at the following molar ratio. The first composition may be produced, for example, by the enzyme treatment described above, or the enzyme treatment and subsequent silver treatment described above.

3.0≦(lower alcohol ester of EPA/lower alcohol ester of DHA)≦30

The molar ratio is more preferably the following molar ratio.

3.0≦(lower alcohol ester of EPA/lower alcohol ester of DHA)≦20

The molar ratio is preferably the following molar ratio.

3.0≦(lower alcohol ester of EPA/lower alcohol ester of DHA)≦15

In the first composition, a ratio of the intensity of a peak which appears around 966 cm⁻¹ to the intensity of a peak which appears around 1,736 cm⁻¹ in FT-IR spectral analysis may be 0.15 or less (preferably0.13 or less). Thus, in the first composition, an isomerized compound is reduced. When the first composition is subjected to the molecular distillation described above, a second composition having less amount of an isomerized compound (mixture of a lower alcohol ester of EPA and a lower alcohol ester of DHA) can be produced. In addition, when the first composition is subjected to the silver treatment, the composition may have a reduced content of a free fatty acid and an acid value of less than 5 (preferably less than 4).

<Molecular Distillation of First Composition>

A method of producing a lower alcohol ester of fatty acid-containing composition according to one embodiment of the present invention includes distilling the first composition to separate a mixture of lower alcohol ester of fatty acids including the lower alcohol ester of EPA and the lower alcohol ester of DHA from components other than the mixture, to thereby obtain a second composition described below.

In the production method according to the embodiment, the step of distilling the first composition to obtain a second composition described below corresponds to the molecular distillation step described above.

The production method according to the embodiment includes distilling the first composition to obtain a second composition described below, and hence a lower alcohol ester of EPA can be selectively produced in the rectification.

<Second Composition>

A lower alcohol ester of fatty acid-containing composition according to one embodiment of the present invention (hereinafter sometimes referred to as “second composition”) includes 90 mass % or more (more specifically 95 mass % or more and 100 mass % or less) of a lower alcohol ester of fatty acid, and the lower alcohol ester of fatty acid includes a lower alcohol ester of EPA and a lower alcohol ester of DHA at the following molar ratio. The second composition may be produced, for example, by the molecular distillation treatment described above, or the molecular distillation treatment and subsequent silver treatment described above.

3.0≦(lower alcohol ester of EPA/lower alcohol ester of DHA)≦30

The molar ratio is more preferably the following molar ratio.

3.0≦(lower alcohol ester of EPA/lower alcohol ester of DHA)≦20

The molar ratio is still more preferably the following molar ratio.

3.0≦(lower alcohol ester of EPA/lower alcohol ester of DHA)≦15

In the second composition, a ratio of the intensity of a peak which appears around 966 cm⁻¹ to the intensity of a peak which appears around 1,736 cm⁻¹ in FT-IR spectral analysis may be 0.075 or less (preferably 0.070 or less). Thus, in the second composition, an amount of an isomerized compound is reduced. When the second composition is subjected to the rectification (secondary distillation) described above, a lower alcohol EPA ester composition having less amount of an isomerized compound can be produced.

In addition, the second composition may have a reduced content of a free fatty acid and an acid value of less than 5 (preferably less than 4). A content of a free fatty acid may be reduced by the silver treatment described above. The second composition can be used as, for example, a raw material of a food composition or a capsule formulation, such as a supplement.

<Rectification of Second Composition>

A method of producing a lower alcohol ester of fatty acid-containing composition according to one embodiment of the present invention includes distilling the second composition to separate a lower alcohol ester of fatty acid other than the lower alcohol ester of EPA, to thereby obtain a lower alcohol ester of EPA (third composition described below).

In the production method according to the embodiment, distilling the second composition to obtain a lower alcohol ester of EPA corresponds to the rectification step described above.

The production method according to the embodiment includes distilling the second composition to obtain a lower alcohol ester of EPA. Thus, a lower alcohol ester of EPA can be selectively produced. A lower alcohol ester of EPA having high purity can be used, for example, as a raw material of a food composition or a capsule formulation, such as a supplement.

<Third Composition>

A lower alcohol ester of fatty acid-containing composition according to one embodiment of the present invention (hereinafter sometimes referred to as “third composition”) includes 96.5 mass % or more (more preferably 98 mass % or more and about 100 mass % or less) of a lower alcohol ester of EPA and has, in FT-IR spectral analysis of the composition, a ratio of the intensity of a peak which appears around 966 cm⁻¹ to the intensity of a peak which appears around 1,736 cm⁻¹ of preferably 0.085 or less.

The third composition may be produced through the rectification step. The third composition has extremely less content of an isomerized compound and hence can be suitably used, for example, as a raw material of a food composition or a capsule formulation, such as a supplement.

### EXAMPLES

The present invention is hereinafter described in more detail by way of Examples. However, the present invention is by no means limited to Examples.

### Preparation Example 1

### Preparation of Immobilized Enzyme

A solution of Thermomyces lanuginose was sprayed in an amount of 400 g (940 KLU/mL) onto 1 kg of Celite 545 (Johns-Manville Corporation, particle size of from 0.02 mm to 0.1 mm) using a fluidized bed granulator manufactured by Okawara Mfg. Co., Ltd. The lipase solution was supplied through a peristaltic pump (manufactured by Tokyo Rikakikai Co., Ltd.). The temperature of air in an intake port from air stream at 100 m³/hr was 57° C. and the temperature of the immobilized product was about 40° C. After completion of immobilization, the immobilized product was further dried in the fluidized bed for 5 minutes to obtain a particulate immobilized enzyme (average particle size of 600 μm, specific gravity of 2).

### Example 1

### Enzyme Treatment

A separable flask (volume of 3 L) was charged with 1 kg of a refined fish oil (sardine oil, acid value of 0) and 52.5 g of ethanol was added thereto. The flask was shaken to uniformly disperse the ethanol in the fish oil. Then, 21 g of water (a content of water in the reaction solution: 2 mass %) was charged thereto and stirred to disperse water in the fish oil-ethanol mixture and a reaction solution was prepared. Then, 105 g of the immobilized enzyme prepared in Example 1 was added thereto, and atmospheric air in the sample bottle was replaced with nitrogen before the sample was subjected to a reaction at 150 rpm. and 30° C. for 24 hours using a stirrer to obtain a lower alcohol ester of fatty acid-containing composition including EPAEE and DHAEE (first composition). At each time point of 0 hours, 2 hours, 4 hours, 6 hours, and 24 hours from the beginning of the reaction, 200 μl each of the reaction solution was collected and subjected to component analysis (a content of the lower alcohol ester of fatty acid in a lower alcohol ester of fatty acid-containing composition (mass %), a content of EPAEE in the lower alcohol ester of fatty acid (mol %), a content of DHAEE in the lower alcohol ester of fatty acid (mol %), and a ratio EPAEE/DHAEE (molar ratio). In addition, at each time point of 2 hours, 4 hours, and 6 hours from the beginning of the reaction, 52.5 g of ethanol was added to the reaction solution and the sample bottle was purged with nitrogen. In addition, the reaction from the beginning to 24 hours was defined as 1 cycle and the reaction was repeatedly performed in 3 cycles. After completion of each cycle, the reaction solution was suctioned and filtered to divide into oil and an immobilized enzyme and the divided immobilized enzyme was transferred into a reaction container. Then, a required amount of oil was added to the container, the mixture was repeatedly used in the reaction in subsequent cycles. In addition, at each time point of 0 hours, 2 hours, 4 hours, 6 hours, 8 hours, and 24 hours (completion of the reaction) from the beginning of the reaction, the reaction solution was collected in a trace amount and subjected to component analysis.

The acid value of the reaction solution in Example 1 (first cycle of the reaction from the beginning to 24 hours) and the acid value of the reaction solution in Example 1 (third cycle of the reaction from the beginning to 8 hours) were measured and calculated by the method in the above-mentioned embodiment and the acid values were 5.95 and 6.17, respectively.

In addition, in FT-IR spectral analysis of the reaction solution in Example 1 (first cycle of the reaction from the beginning to 24 hours) (measurement apparatus: a single reflection-type total reflection measurement apparatus MIRacleA (ZnSe prism)), a ratio of the intensity of a peak which appeared around 966 cm⁻¹ to the intensity of a peak which appeared around 1,736 cm⁻¹ was 0.12.

### Examples 2 to 8

### Enzyme Treatment

Lower alcohol ester of fatty acid-containing compositions of Examples 2 to 8 including EPAEE and DHAEE (first compositions) were produced in the same manner as that in Example 1 other than that an amount of water to be used was 5.25 g (a content of water in the reaction solution: 0.5 mass %), 10.5 g (a content of water in the reaction solution: 1 mass %), 52.5 g (a content of water in the reaction solution: 5 mass %), 105 g (a content of water in the reaction solution: 9 mass %), 210 g (a content of water in the reaction solution: 17 mass %), 525 g (a content of water in the reaction solution: 33 mass %), or 1,050 g (a content of water in the reaction solution: 50 mass %).

In Examples 3 to 5, similarly to Example 1, a reaction from the beginning to 24 hours was defined as 1 cycle and the reaction was repeatedly performed in 3 cycles. After completion of each cycle, the reaction solution was suctioned and filtered to divide into oil and an immobilized enzyme and the divided immobilized enzyme was transferred into a reaction container. Then, a required amount of oil was added to the container, and the mixture was repeatedly used in the reaction in subsequent cycles.

Acid values of the reaction solutions in Examples 2 to 8 were measured and calculated by the method in the above-mentioned embodiment and each of the acid values fell within a range of from 2.2 or more to 12 or less.

In addition, in FT-IR spectral analysis of the reaction solutions in Examples 2 to 8 (measurement apparatus: single reflection-type total reflection measurement apparatus MIRacleA (ZnSe prism)), a ratio of the intensity of a peak which appeared around 966 cm⁻¹ to the intensity of a peak which appeared around 1,736 cm⁻¹ was 0.15 or less in each of Examples described above.

### Comparative Example 1

### Enzyme Treatment

A lower alcohol ester of fatty acid-containing composition of Comparative Example 1 including EPAEE and DHAEE was produced in the same manner as that in Example 1 other than that an amount of water to be used was 0 g (a content of water in the reaction solution: 0%).

### Comparative Example 2

### Enzyme Treatment

A lower alcohol ester of fatty acid-containing composition of Comparative Example 2 including EPAEE and DHAEE was produced in the same manner as that in Example 1 except that an amount of water to be used was 3.15 g (a content of water in the reaction solution: 0.3 mass %).

The acid values of the reaction solutions in Comparative Examples 1 and 2 were measured and calculated by the method in the above-mentioned embodiment and the acid value of the reaction solution in Comparative Example 1 was 1.0 and the acid value of the reaction solution in Comparative Example 2 was 1.5.

Contents of lower alcohol ester of fatty acids in lower alcohol ester of fatty acid-containing compositions (mass %), contents of EPAEE in lower alcohol ester of fatty acids (mol %), contents of DHAEE in lower alcohol ester of fatty acids (mol %), and the ratio EPAEE/DHAEE (molar ratio) in the lower alcohol ester of fatty acid-containing compositions of Examples 1 to 8 and Comparative Examples 1 and 2 are shown in Table 1.

TABLE 1
Example 1 (Water content: 2 mass %)

| | | Content of lower | Content | Content | EPA |
|---|---|---|---|---|---|
| | Reaction | alcohol ester | of EPA | of DHA | ethyl ester/ |
| | time | of fatty acid | ethyl ester | ethyl ester | DHA |
| | (hrs) | (%) | (%) | (%) | ethyl ester |
| First | 2 | 15.6 | 18.7 | 2.2 | 8.5 |
| cycle | 4 | 32.8 | 18.2 | 2.3 | 7.9 |
| | 6 | 40.8 | 18.7 | 2.8 | 6.7 |
| | 8 | 50.9 | 20.5 | 3.5 | 5.9 |
| | 24 | 69.9 | 20.5 | 5.6 | 3.7 |
| Second | 2 | 25.9 | 17.5 | 3.2 | 5.5 |
| cycle | 4 | 37.6 | 18.8 | 3.3 | 5.7 |
| | 6 | 44.9 | 19.5 | 3.5 | 5.6 |
| | 8 | 49.5 | 20.6 | 3.8 | 5.4 |
| | 24 | 67.5 | 20.9 | 5.7 | 3.7 |
| Third | 2 | 23.0 | 17.3 | 2.8 | 6.2 |
| cycle | 4 | 37.8 | 18.9 | 3.0 | 6.3 |
| | 6 | 47.3 | 19.7 | 3.3 | 6.0 |
| | 8 | 55.1 | 20.7 | 3.7 | 5.6 |

Example 2 (Water content: 0.5 mass %)

| | | Content of lower | Content | Content | EPA |
|---|---|---|---|---|---|
| | Reaction | alcohol ester | of EPA | of DHA | ethyl ester/ |
| | time | of fatty acid | ethyl ester | ethyl ester | DHA |
| | (hrs) | (%) | (%) | (%) | ethyl ester |
| First | 2 | 19.5 | 15.0 | 1.6 | 9.5 |
| cycle | 4 | 34.0 | 15.8 | 2.5 | 6.5 |
| | 6 | 42.8 | 16.8 | 2.4 | 7.0 |
| | 8 | 46.4 | 17.7 | 2.6 | 6.8 |
| | 23 | 58.9 | 18.8 | 3.5 | 5.4 |
| Second | 2 | 17.7 | 16.5 | 2.3 | 7.1 |
| cycle | 4 | 35.4 | 16.6 | 2.3 | 7.1 |
| | 6 | 36.1 | 16.9 | 2.4 | 7.0 |
| | 8 | 33.2 | 17.4 | 2.3 | 7.4 |
| | 23 | 39.9 | 19.1 | 2.9 | 6.6 |
| Third | 2 | 20.6 | 15.5 | 2.3 | 6.8 |
| cycle | 4 | 25.4 | 16.2 | 2.2 | 7.5 |
| | 6 | 30.5 | 16.5 | 2.1 | 7.8 |
| | 8 | 32.0 | 17.0 | 2.2 | 7.8 |
| | 23 | 56.3 | 19.2 | 2.9 | 6.7 |

Example 3 (Water content: 1 mass %)

| | | Content of lower | Content | Content | EPA |
|---|---|---|---|---|---|
| | Reaction | alcohol ester | of EPA | of DHA | ethyl ester/ |
| | time | of fatty acid | ethyl ester | ethyl ester | DHA |
| | (hrs) | (%) | (%) | (%) | ethyl ester |
| First | 2 | 18.5 | 18.1 | 2.0 | 9.1 |
| cycle | 4 | 31.2 | 18.2 | 2.3 | 7.9 |
| | 6 | 41.4 | 18.7 | 2.7 | 6.9 |
| | 8 | 43.1 | 20.3 | 3.3 | 6.2 |
| | 24 | 65.7 | 20.5 | 4.8 | 4.3 |
| Second | 2 | 12.9 | 18.1 | 3.6 | 5.0 |
| cycle | 4 | 24.0 | 18.3 | 3.4 | 5.4 |
| | 6 | 25.7 | 19.1 | 3.5 | 5.5 |
| | 8 | 33.4 | 20.5 | 3.7 | 5.5 |
| | 24 | 51.8 | 20.7 | 4.3 | 4.8 |
| Third | 2 | 14.5 | 17.1 | 2.7 | 6.3 |
| cycle | 4 | 25.1 | 17.7 | 2.6 | 6.8 |
| | 6 | 33.4 | 18.8 | 2.7 | 7.0 |
| | 8 | 49.8 | 19.9 | 2.8 | 7.1 |

Example 4 (Water content: 5 mass %)

| | | Content of lower | Content | Content | EPA |
|---|---|---|---|---|---|
| | Reaction | alcohol ester | of EPA | of DHA | ethyl ester/ |
| | time | of fatty acid | ethyl ester | ethyl ester | DHA |
| | (hrs) | (%) | (%) | (%) | ethyl ester |
| First | 2 | 16.5 | 18.5 | 2.2 | 8.4 |
| cycle | 4 | 34.8 | 18.1 | 2.3 | 7.9 |
| | 6 | 42.9 | 19.0 | 2.8 | 6.8 |
| | 8 | 52.2 | 20.4 | 3.5 | 5.8 |
| | 24 | 70.6 | 20.7 | 6.3 | 3.3 |
| Second | 2 | 31.2 | 18.2 | 3.1 | 5.9 |
| cycle | 4 | 41.4 | 19.8 | 3.3 | 6.0 |
| | 6 | 57.1 | 20.2 | 3.6 | 5.6 |
| | 8 | 55.4 | 21.1 | 4.2 | 5.0 |
| | 24 | 73.0 | 20.8 | 6.2 | 3.4 |
| Third | 2 | 29.5 | 18.0 | 3.0 | 6.0 |
| cycle | 4 | 50.3 | 19.7 | 3.3 | 6.0 |
| | 6 | 56.4 | 20.1 | 3.7 | 5.4 |
| | 8 | 60.0 | 21.1 | 4.1 | 5.1 |

Example 5 (Water content: 9 mass %)

| | | Content of lower | Content | Content | EPA |
|---|---|---|---|---|---|
| | Reaction | alcohol ester | of EPA | of DHA | ethyl ester/ |
| | time | of fatty acid | ethyl ester | ethyl ester | DHA |
| | (hrs) | (%) | (%) | (%) | ethyl ester |
| First | 2 | 14.9 | 18.6 | 2.2 | 8.5 |
| cycle | 4 | 41.0 | 18.6 | 2.3 | 8.1 |
| | 6 | 41.0 | 19.3 | 2.8 | 6.9 |
| | 8 | 50.8 | 20.4 | 3.5 | 5.8 |
| | 24 | 69.4 | 20.6 | 6.2 | 3.3 |
| Second | 2 | 22.4 | 16.6 | 2.8 | 5.9 |
| cycle | 4 | 33.7 | 18.5 | 2.9 | 6.4 |
| | 6 | 47.9 | 19.6 | 3.3 | 5.9 |
| | 8 | 46.6 | 20.9 | 3.7 | 5.6 |
| | 24 | 75.5 | 20.8 | 6.0 | 3.5 |
| Third | 2 | 18.1 | 16.7 | 2.9 | 5.8 |
| cycle | 4 | 33.0 | 18.5 | 3.1 | 6.0 |
| | 6 | 42.3 | 19.7 | 3.4 | 5.8 |
| | 8 | 56.3 | 20.3 | 3.8 | 5.3 |

Example 6 (Water content: 17 mass %)

| | | Content of lower | Content | Content | EPA |
|---|---|---|---|---|---|
| | Reaction | alcohol ester | of EPA | of DHA | ethyl ester/ |
| | time | of fatty acid | ethyl ester | ethyl ester | DHA |
| | (hrs) | (%) | (%) | (%) | ethyl ester |
| First | 2 | 16.2 | 20.0 | 1.7 | 11.8 |
| cycle | 6 | 50.2 | 19.4 | 2.4 | 8.1 |
| | 8 | 60.4 | 19.4 | 2.9 | 6.7 |
| | 24 | 74.9 | 19.4 | 5.4 | 3.6 |

Example 7 (Water content: 33 mass %)

| | | Content of lower | Content | Content | EPA |
|---|---|---|---|---|---|
| | Reaction | alcohol ester | of EPA | of DHA | ethyl ester/ |
| | time | of fatty acid | ethyl ester | ethyl ester | DHA |
| | (hrs) | (%) | (%) | (%) | ethyl ester |
| First | 2 | 11.9 | 20.8 | 1.8 | 11.6 |
| cycle | 6 | 43.9 | 19.0 | 2.0 | 9.5 |
| | 8 | 56.7 | 19.2 | 2.4 | 8.0 |
| | 24 | 72 | 20.0 | 5.2 | 3.8 |

Example 8 (Water content: 50 mass %)

| | | Content of lower | Content | Content | EPA |
|---|---|---|---|---|---|
| | Reaction | alcohol ester | of EPA | of DHA | ethyl ester/ |
| | time | of fatty acid | ethyl ester | ethyl ester | DHA |
| | (hrs) | (%) | (%) | (%) | ethyl ester |
| First | 2 | 8.6 | 21.2 | 1.7 | 12.5 |
| cycle | 6 | 34.1 | 20.0 | 1.9 | 10.5 |
| | 8 | 38.7 | 19.3 | 2.0 | 9.7 |
| | 24 | 64.4 | 21.7 | 5.2 | 4.2 |

Comparative Example 1 (Water content: 0 mass %)

| | | Content of lower | Content | Content | EPA |
|---|---|---|---|---|---|
| | Reaction | alcohol ester | of EPA | of DHA | ethyl ester/ |
| | time | of fatty acid | ethyl ester | ethyl ester | DHA |
| | (hrs) | (%) | (%) | (%) | ethyl ester |
| First | 2 | 12.0 | 11.9 | 1.0 | 11.9 |
| cycle | 4 | 17.3 | 12.5 | 1.0 | 12.5 |
| | 6 | 22.6 | 13.3 | 1.1 | 12.1 |
| | 8 | 22.3 | 14.0 | 1.1 | 12.7 |
| | 24 | 32.1 | 17.1 | 1.5 | 11.4 |
| Second | 2 | 6.9 | 15.5 | 1.6 | 9.7 |
| cycle | 4 | 10.2 | 14.4 | 1.4 | 10.3 |
| | 6 | 9.8 | 13.4 | 1.3 | 10.3 |
| | 8 | 11.8 | 13.1 | 1.2 | 10.9 |
| | 24 | 17.8 | 12.4 | 1.1 | 11.3 |
| Third | 2 | 4.1 | 12.2 | 1.1 | 11.1 |
| cycle | 4 | 6.9 | 11.4 | 1.2 | 9.5 |
| | 6 | 7.5 | 11.1 | 1.2 | 9.3 |
| | 8 | 35.4 | 11.1 | 1.1 | 10.1 |

Comparative Example 2 (Water content: 0.3 mass %)

| | | Content of lower | Content | Content | EPA |
|---|---|---|---|---|---|
| | Reaction | alcohol ester | of EPA | of DHA | ethyl ester/ |
| | time | of fatty acid | ethyl ester | ethyl ester | DHA |
| | (hrs) | (%) | (%) | (%) | ethyl ester |
| First | 2 | 18.8 | 12.9 | 1.2 | 11.3 |
| cycle | 4 | 29.2 | 14.7 | 1.5 | 9.7 |
| | 6 | 34.9 | 16.1 | 1.7 | 9.7 |
| | 8 | 40.6 | 17.1 | 1.7 | 10.3 |
| | 23 | 43.3 | 18.4 | 2.2 | 8.3 |
| Second | 2 | 15.0 | 14.6 | 2.1 | 7.0 |
| cycle | 4 | 20.6 | 13.8 | 1.9 | 7.4 |
| | 6 | 24.3 | 13.7 | 1.8 | 7.6 |
| | 8 | 22.4 | 13.7 | 1.5 | 9.3 |
| | 23 | 24.1 | 14.4 | 1.7 | 8.7 |
| Third | 2 | 11.2 | 11.6 | 1.2 | 9.6 |
| cycle | 4 | 13.5 | 11.4 | 1.2 | 9.8 |
| | 6 | 16.7 | 11.5 | 1.2 | 9.8 |
| | 8 | 18.3 | 11.7 | 1.2 | 10.0 |
| | 23 | 25.3 | 14.0 | 1.3 | 11.1 |

From Table 1, it can be found that in Examples 1 to 8, contents of EPAEE in lower alcohol ester of fatty acids sharply increase in the reaction from the beginning to 2 hours, and as the reaction time increases, contents of lower alcohol ester of fatty acids in the reaction solution tend to increase and also contents of EPAEE and DHAEE in lower alcohol ester of fatty acids tend to increase, resulting in that values of contents of EPAEE/contents of DHAEE in the reaction solutions increase compared to values of EPA/DHA constituting a fatty acid glyceride included in a raw material oil and fat before reactions.

In addition, it can be found that in Examples 1 to 8, when each of contents of water in the reaction solutions in the enzyme treatment is 0.4 mass % or more, the stability of the enzyme is enhanced and hence the enzyme can be repeatedly used, resulting in that the immobilized enzyme can be removed from the reaction solution and the removed enzyme can be used again.

In addition, in treatments with a lipase according to Examples 1 to 8, when each of contents of water in the reaction solutions was 0.4 mass % or more, it was possible to obtain a lower alcohol ester of fatty acid-containing composition having a content of a lower alcohol ester of fatty acid of 40 mass % or more and 90 mass % or less, and a molar ratio of the EPAEE to DHAEE (EPAEE/DHAEE) of 3.0 or more and 30 or less.

In a fatty acid constituting a fatty acid glyceride included in a raw material oil and fat before the reaction, a content of EPA and a content of DHA are 18 mol % and 12 mol % (EPA/DHA=3/2), respectively. As described above, it can be found that through the treatment described above, EPAEE can be selectively produced from a raw material oil and fat including an EPA-containing glyceride.

On the other hand, in treatments with a lipase according to Comparative Examples 1 and 2, when each of contents of water in the reaction solutions was less than 0.4 mass %, the stability of the enzyme was lowered, and the content of the lower alcohol ester of fatty acid in the reaction solution was decreased compared to those in the reaction solutions obtained in Examples 1 to 8.

### Example 9

### Molecular Distillation Treatment

The first composition obtained in Example 1 was distilled at a degree of vacuum of 0.1 Torr or less and a temperature of 80° C. or more and 200° C. or less using a short-path distillation apparatus (manufactured by Kobelco Eco-Solutions Co., Ltd.) (molecular distillation (primary distillation)) to obtain a lower alcohol ester of fatty acid-containing composition including a lower alcohol ester of EPA (EPAEE) and a lower alcohol ester of DHA (DHAEE) (second composition).

In FT-IR spectral analysis of the second composition obtained in Example 9 (measurement apparatus: single reflection-type total reflection measurement apparatus MIRacleA (ZnSe prism)), a ratio of the intensity of a peak which appeared around 966 cm⁻¹ to the intensity of a peak which appeared around 1,736 cm⁻¹ was0.069, the content of the lower alcohol fatty acid ethyl ester included in the second composition was almost 100 mass %, the acid value of the second composition was 10.1, and the ratio EPAEE/DHAEE (molar ratio) in the second composition was 6.0.

### Example 10

### Rectification Treatment

The second composition obtained in Example 9 was distilled at a degree of vacuum of 3 Torr or less, a temperature of 150° C. or more and 250° C. or less, and a number of theoretical plates of 5 using a falling-film type rectification apparatus (manufactured by Asahi Glassplant Inc.) (rectification (secondary distillation)) to obtain EPAEE (third composition, purity: almost 100 mass %).

In FT-IR spectral analysis of EPAEE in Example 10 (measurement apparatus: single reflection-type total reflection measurement apparatus MIRacleA (ZnSe prism)), a ratio of the intensity of a peak which appeared around 966 cm⁻¹ to the intensity of a peak which appeared around 1,736 cm⁻¹ was0.081, the content of the fatty acid ethyl ester (EPAEE) included in the third composition was almost 100 mass %, and the acid value of the third composition was almost 0.

### Comparative Example 3

### Preparation of Lower Alcohol Ester of Fatty Acid-Containing Composition by Alkaline Treatment and Preparation of Mixture of EPAEE and DHAEE

To a 2 L Erlenmeyer flask with a stopper (shielding light with aluminum foil) was added 800 g of the same refined fish oil (a content of water obtained by measurement by Karl Fischer method of 0.04 mass %) as the one used in Example 1. Another beaker was charged with 240 ml of ethanol and 4.8 g of sodium hydroxide was added thereto to prepare a 2% (w/w) solution of sodium hydroxide in ethanol. The 2% (w/w) solution of sodium hydroxide in ethanol was added in the Erlenmeyer flask and then, the Erlenmeyer flask was purged with nitrogen. Subsequently, the Erlenmeyer flask was immersed in a constant temperature water bath at 30° C. and the contents in the flask were stirred at a stirrer scale of 8, and room temperature (25° C.) for 18 hours. Subsequently, the reaction solution was transferred to a separatory funnel, and the funnel was washed by addition of 50 g of pure water. After the funnel was left to stand still for about 20 minutes and then, washing operation was performed to discard a lower phase (water phase). Then, 50 g of pure water was added thereto and the same washing operation was performed. Subsequently, the same washing operation was performed an additional four times (an amount of pure water to be added, first: 50 g, second: 240 g, third: 240 g, fourth: 240 g). Subsequently, the oil phase was confirmed to be neutral and then, anhydrous sodium sulfate was added thereto and the whole was left to stand still overnight. The resultant mixture was stored at 4° C. and a lower alcohol ester of fatty acid-containing composition of Comparative Example 3 was obtained.

The lower alcohol ester of fatty acid-containing composition of Comparative Example 3 was subjected to the same distillation treatment as Example 9 to obtain a mixture of EPAEE and DHAEE.

In FT-IR spectral analysis of the reaction solution in Comparative Example 3 (measurement apparatus: single reflection-type total reflection measurement apparatus MIRacleA (ZnSe prism)), a ratio of the intensity of a peak which appeared around 966 cm⁻¹ to the intensity of a peak which appeared around 1,736 cm⁻¹ was 0.095, and the content of the fatty acid ethyl ester included in the reaction solution was almost 100 mass %.

As described above, the method of producing a lower alcohol ester of fatty acid-containing composition according to the present invention (Example 9) was able to reduce the amount of the isomerized compound included in a lower alcohol ester of fatty acid-containing composition compared to a method involving alkaline treatment (Comparative Example 3).

### Preparation Example 2

### Preparation of Immobilized Enzyme

According to an ordinary method, 70 mass % of divinylbenzene (DVB), 15 mass % of glycidyl methacrylate, and 15 mass % of DEAE methacrylate were copolymerized to obtain a particulate resin carrier. The resin carrier has an average pore size of 11.5 nm, a pore volume of 0.5 cm³/g, an average particle size of 0.5 mm, and a specific gravity of 0.2. To 1 kg of the resultant resin carrier was added 10 L of a 2 mass % aqueous solution of lipase FAP-15 (manufactured by Amano Enzyme Inc., 155,000 u/g) derived from Rhizopus sp., and the mixture was immobilized with stirring at 25° C. for 3 hours. After filtration and washing, the immobilized mixture was dried for 2 hours in a vacuum drier to obtain an immobilized enzyme.

### Examples 11, 12, 13, and 14

### Enzyme Treatment

The reaction was performed in 1 cycle in the same method as Example 1 except that the immobilized enzyme used in the enzyme treatment in Example 1 was replaced with the immobilized enzyme obtained in Preparation Example 2, and an amount of enzyme, an amount of ethanol, and a content of water described in Table 2 were used, and component analysis was performed. In Example 13, at each time point of the beginning of the reaction and 4 hours from the beginning of the reaction, ethanol was added in an amount of 105 g. In Example 14, ethanol was added in an amount of 210 g at the beginning of the reaction.

The acid value of the reaction solution in Example 11 (at a time point of 24 hours from the beginning of the reaction) was measured and calculated by the method in the above-mentioned embodiment and the acid value was 4.6.

TABLE 2

| | | | | | Content | | | |
|---|---|---|---|---|---|---|---|---|
| | | | | | of lower | | | |
| | | | | | alcohol | Content | Content | |
| | Amount | | | | ester of | of EPA | of DHA | EPA ethyl |
| | of | | | Reaction | fatty | ethyl | ethyl | ester/DHA |
| | enzyme | | Water | time | acid | ester | ester | ethyl |
| | used | Amount of ethanol used | content | (hrs) | (mass %) | (mol %) | (mol %) | ester |
| Example | 105 g | The same amount as that in | 2 mass % | 2 | 14.4 | 19.9 | 1.6 | 12.4 |
| 11 | | Example 1 | | 4 | 39.8 | 20.0 | 2.0 | 10.0 |
| | | | | 6 | 56.1 | 20.2 | 2.8 | 7.2 |
| | | | | 8 | 52.9 | 20.1 | 2.5 | 8.0 |
| | | | | 24 | 75.5 | 22.9 | 5.4 | 4.2 |
| Example | 210 g | The same amount as that in | 4 mass % | 2 | 18.0 | 23.0 | 2.3 | 10.0 |
| 12 | | Example 1 | | 4 | 48.3 | 20.4 | 3.2 | 6.4 |
| | | | | 6 | 72.5 | 19.9 | 4.4 | 4.5 |
| | | | | 8 | 65.2 | 19.8 | 4.5 | 4.4 |
| | | | | 24 | 82.0 | 19.1 | 5.0 | 3.8 |
| Example | 210 g | At each time point of 0 | 4 mass % | 2 | 47.3 | 20.0 | 2.8 | 7.1 |
| 13 | | hour and 4 hours from the | | 4 | 46.8 | 20.2 | 3.4 | 5.9 |
| | | beginning of the reaction, | | 6 | 61.5 | 20.2 | 3.9 | 5.2 |
| | | ethanol was added in an | | 8 | 63.4 | 19.8 | 3.9 | 5.1 |
| | | amount of 105 g. | | 24 | 79.8 | 24.0 | 8.1 | 3.0 |
| Example | 210 g | At a time point of the | 0.5 mass % | 2 | 17.8 | 14.5 | 1.0 | 14.5 |
| 14 | | beginning of the reaction, | | 4 | 20.6 | 16.3 | 1.3 | 12.5 |
| | | ethanol was added in an | | 6 | 24.7 | 18.3 | 2.0 | 9.2 |
| | | amount of 210 g. | | 8 | 31.2 | 18.9 | 2.3 | 8.2 |
| | | | | 24 | 54.4 | 19.2 | 4.8 | 4.0 |

From Table 2, it can be found that in Examples 11 to 14, contents of EPAEE in lower alcohol ester of fatty acids sharply increase in 2 hours from the beginning of the reaction, and as the reaction time increases, contents of lower alcohol ester of fatty acids in the reaction solution tend to increase and also contents of EPAEE and DHAEE in lower alcohol ester of fatty acids tend to increase, resulting in that values of contents of EPAEE/contents of DHAEE in the reaction solutions increase compared to values of EPA/DHA constituting a fatty acid glyceride included in a raw material oil and fat before reactions.

In addition, according to Examples 11 to 14, in treatments with a lipase, when each of contents of water in the reaction solutions was 0.4 mass % or more, it was possible to obtain a lower alcohol ester of fatty acid-containing composition having a content of a lower alcohol ester of fatty acid of 40 mass % or more and 90 mass % or less, and a molar ratio of the EPAEE to DHAEE (EPAEE/DHAEE) of 3.0 or more and 15.0 or less.

In addition, in FT-IR spectral analysis of the reaction solutions in Examples 11 to 14 (measurement apparatus: single reflection-type total reflection measurement apparatus MIRacleA (ZnSe prism)), a ratio of the intensity of a peak which appeared around 966 cm⁻¹ to the intensity of a peak which appeared around 1,736 cm⁻¹ was 0.15 or less.

### Example 15

### Silver Treatment

The lower alcohol ester of fatty acid-containing composition obtained in Example 9 (second composition) was mixed in an amount of 10 g with 40 g of an aqueous silver salt solution (a concentration of silver nitrate: 50 mass %) under a nitrogen atmosphere and light shielding at 20° C. for 20 minutes, to bring the composition into contact with the aqueous silver salt solution (test No. 1 in Table 3). In addition, the same treatment was performed with changed amounts of the aqueous silver salt solution to be used (test Nos. 2 and 3 in Table 3).

After the contact, the separated organic phase was discarded, 40 g of toluene was added to the remainder of the aqueous silver salt solution, and then the mixture was stirred at 60° C. for 1 hour. The toluene layer including EPAEE and DHAEE was collected and then, the toluene was removed from the layer to obtain a mixture of EPAEE and DHAEE.

Acid values and values of a content of EPAEE/a content of DHAEE (molar ratio) before and after silver treatment were shown in Table 3.

TABLE 3

| | | Kind of silver | | EPA |
|---|---|---|---|---|
| | | salt and amount | | ethyl ester/ |
| | | of silver | Acid | DHA |
| | Test No. | nitrate used | value | ethyl ester |
| Before | Raw material | — | 10.1 | 12.1 |
| silver | (second | | | |
| treatment | composition) | | | |
| Silver | Test No. 1 | 20 g of silver | 3.9 | 13.9 |
| treatment | | nitrate | | |
| groups | Test No. 2 | 30 g of silver | 2.5 | 13.3 |
| | | nitrate | | |
| | Test No. 3 | 50 g of silver | 1.9 | 13.0 |
| | | nitrate | | |

As shown in Table 3, it can be found that through silver treatment in Examples of the present invention, while a ratio of EPA ethyl ester/DHA ethyl ester was kept, acid values of the lower alcohol ester of fatty acid-containing composition (second composition) were able to be reduced to less than 5. In the EPAEE obtained in Example 10, the same silver treatment as that in this Example was performed and through the treatment, it was possible to remove a trace amount of a free fatty acid therein.

### Example 16

### Rectification Treatment

The second composition after silver treatment obtained in Example 15 was distilled at a degree of vacuum of 3 Torr or less, a temperature of 150° C. or more and 200° C. or less, and a number of theoretical plates of 5 using a rectification apparatus (manufactured by Asahi Glassplant Inc.) (rectification) to obtain EPAEE (third composition, purity: almost 100 mass %, acid value: almost 0).

In FT-IR spectral analysis of EPAEE in Example 16 (measurement apparatus: single reflection-type total reflection measurement apparatus MIRacleA (ZnSe prism)), a ratio of the intensity of a peak which appeared around 966 cm⁻¹ to the intensity of a peak which appeared around 1,736 cm⁻¹ was0.078, the content of the fatty acid ethyl ester included in the third composition was almost 100 mass %.

### Example 17

### Enzyme Treatment, Molecular Distillation Treatment, and Rectification Treatment

The enzyme treatment in Example 1, the molecular distillation treatment in Example 9 and the rectification treatment in Example 10 were performed at scales of 1,000 times, 2,000 times, and 2,000 times, respectively. As a result, the component of the first composition obtained in the enzyme treatment of this Example (a content of the lower alcohol fatty acid ester in the lower alcohol ester of fatty acid (mass %), the content of EPAEE in the lower alcohol ester of fatty acids (mol %), the content of DHAEE in the lower alcohol ester of fatty acids (mol %), the ratio EPAEE/DHAEE (molar ratio)), the ratio of the intensity of a peak which appeared around 966 cm⁻¹ to the intensity of a peak which appeared around 1,736 cm⁻¹ in FT-IR spectral analysis of the reaction solution (mixture), and the acid value were the same as those in the first composition obtained in Example 1.

In addition, the content of the lower alcohol fatty acid ethyl ester included in the second composition obtained in molecular distillation treatment in this Example and the ratio EPAEE/DHAEE (molar ratio) were the same as those in the second composition obtained in Example 9. In addition, the ratio of the intensity of a peak which appeared around 966 cm⁻¹ to the intensity of a peak which appeared around 1,736 cm⁻¹ in FT-IR spectral analysis of the second composition obtained by molecular distillation treatment in this Example and the acid value were the same as those in the second composition obtained in Example 9.

Further, the content of EPAEE included in the third composition obtained in rectification treatment in this Example was the same as that in the third composition obtained in Example 10. In addition, the ratio of the intensity of a peak which appeared around 966 cm⁻¹ to the intensity of a peak which appeared around 1,736 cm⁻¹ in FT-IR spectral analysis of the third composition obtained by rectification treatment in this Example and the acid value were the same as those in the third composition obtained in Example 10.

### Example 18

### Food Composition: Cookie

A cookie was prepared with the following blend. A shortening and the lower alcohol ester of fatty acid-containing composition obtained in Example 9 were put into a stirrer (KitchenAid K5SS manufactured by KitchenAid) and mixed together at speed controller lever 6 for 1 minute to obtain a creamy product. To the creamy product were added powdered whole egg and sugar and mixing was performed. Then, to the mixture was gradually added fresh water so as to adjust a specific gravity to 0.8 g/ml. To the resultant mixture were added flour and baking powder which were premixed and then sieved and then, the mixture was stirred for 30 seconds to prepare a dough. The obtained dough was stored in a refrigerator for 2 hours and then, the dough was stretched to a thickness of from about 3 mm to about 5 mm, stripped, and baked in an oven at 180° C. for from 13 minutes to 15 minutes to obtain a cookie.

<Blend>

| | | |
|---|---|---|
| Flour | 200 | g |
| Baking powder | 1 | g |
| Lower alcohol ester of fatty acid- | 1 | g |
| containing composition (Example 9) | | |
| Shortening | 120 | g |
| Caster sugar | 80 | g |
| Powdered whole egg | 12 | g |
| Fresh water | 24 | g |
| Total | 438 | g |

### Example 19

### Soft Capsule

A soft capsule having contents in the following blend was produced using the lower alcohol ester of fatty acid-containing composition obtained in Example 10.

<Blending Ratio>

| | |
|---|---|
| EPAEE (Example 10) | 20% |
| Olive oil | 50% |
| Beeswax | 10% |
| Medium chain fatty acid triglyceride | 10% |
| Emulsifier | 10% |
| Total | 100% |

1. A method of producing a lower alcohol ester of fatty acid-containing composition,
the method comprising treating a raw material oil and fat containing an EPA-containing glyceride with a lipase to obtain a lower alcohol ester of fatty acid-containing composition including a lower alcohol EPA ester,
wherein a content of water in a reaction solution in the treating is 0.4 mass % or more.

2. The method of producing a lower alcohol ester of fatty acid-containing composition according to claim 1,
wherein the reaction solution in the treating further comprises 0.1 part by mass or more and 2.5 parts by mass or less of a lower alcohol relative to 9.5 parts by mass of the raw material oil and fat.

3. The method of producing a lower alcohol ester of fatty acid-containing composition according to claim 2,
wherein the treating comprises adding the lower alcohol to the reaction solution in the treating in a continuous or step-wise manner.

4-5. (canceled)

6. The method of producing a lower alcohol ester of fatty acid-containing composition according to claim 1,
wherein the lipase comprises a 1,3-position-specific lipase.

7. The method of producing a lower alcohol ester of fatty acid-containing composition according to claim 1,
wherein the reaction solution has an acid value of 2 or more.

8. The method of producing a lower alcohol ester pf fatty acid-containing composition according to claim 1,
wherein the raw material oil and fat further contains a DHA-containing glyceride,
wherein the lower alcohol ester of fatty acid-containing composition further includes a lower alcohol ester of DHA, and
wherein a lower alcohol ester of fatty acid included in the lower alcohol ester of fatty acid-containing composition has a molar ratio A of the lower alcohol ester of EPA to the lower alcohol ester of DHA (lower alcohol ester of EPA /lower alcohol ester of DHA) higher than a molar ratio B of EPA to DHA (EPA/DHA) in a fatty acid constituting a fatty acid glyceride included in the raw material oil and fat
wherein the molar ratio A and the molar ratio B have a relationship represented by the following expression (1):
1.5≦A/B (1).

9. (canceled)

10. The method of producing a lower alcohol ester of fatty acid-containing composition according to claim 8,
wherein the lower alcohol ester of fatty acid-containing composition comprises 40 mass % or more and 90 mass % or less of a lower alcohol ester of fatty acid, and
wherein the lower alcohol ester of fatty acid has a molar ratio of the lower alcohol ester of EPA to the lower alcohol ester of DHA (lower alcohol ester of EPA/lower alcohol ester of DHA) of 3.0 or more and 30 or less.

11. The method of producing a lower alcohol ester of fatty acid-containing composition according to claim 7,
wherein the lower alcohol ester of fatty acid-containing composition further includes a lower alcohol ester of DHA, and
wherein the method further comprises distilling the lower alcohol ester of fatty acid-containing composition to separate a mixture of lower alcohol ester of fatty acids including the lower alcohol ester of EPA and the lower alcohol ester of DHA from a component other than the mixture.

12. The method of producing a lower alcohol ester of fatty acid-containing composition according to claim 11,
wherein the mixture has a ratio of an intensity of a peak which appears around 966 cm⁻¹ to an intensity of a peak which appears around 1,736 cm⁻¹ of 0.075 or less in FT-IR spectral analysis thereof.

13. The method of producing a lower alcohol ester of fatty acid-containing composition according to claim 11,
wherein the mixture comprises 90 mass % or more of a lower alcohol ester of fatty acid, and
wherein the lower alcohol ester of fatty acid comprises a lower alcohol ester of EPA and a lower alcohol ester of DHA at the following molar ratio:
3.0≦(lower alcohol ester of EPA/lower alcohol ester of DHA)≦30.

14. The method of producing a lower alcohol ester of fatty acid-containing composition according to claim 11, the method further comprising bringing the mixture into contact with an aqueous silver salt solution.

15. The method of producing a lower alcohol ester of fatty acid-containing composition according to claim 14, the method further comprising distilling the mixture after being brought into contact with the aqueous silver salt solution to separate a lower alcohol ester of fatty acid other than the lower alcohol ester of EPA, to thereby obtain the lower alcohol ester of EPA.

16. The method of producing a lower alcohol ester of fatty acid-containing composition according to claim 11, the method further comprising distilling the mixture to separate a lower alcohol ester of fatty acid other than the lower alcohol ester of EPA, to thereby obtain the lower alcohol ester of EPA.

17. A lower alcohol ester of fatty acid-containing composition, comprising 40 mass % or more and 90 mass % or less of a lower alcohol ester of fatty acid,
wherein the lower alcohol ester of fatty acid comprises a lower alcohol ester of EPA and a lower alcohol ester of DHA at the following molar ratio:
3.0≦(lower alcohol ester of EPA/lower alcohol ester of DHA)≦30.

18. The lower alcohol ester of fatty acid-containing composition according to claim 17,
wherein the lower alcohol ester of fatty acid-containing composition has a ratio of an intensity of a peak which appears around 966 cm⁻¹ to an intensity of a peak which appears around 1,736 cm⁻¹ of 0.15 or less in FT-IR spectral analysis thereof.

19. A lower alcohol ester of fatty acid-containing composition, comprising 90 mass % or more of a lower alcohol ester of fatty acid,
wherein the lower alcohol ester of fatty acid comprises a lower alcohol ester of EPA and a lower alcohol ester of DHA at the following molar ratio:
3.0≦(lower alcohol ester of EPA/lower alcohol ester of DHA)≦30.

20. The lower alcohol ester of fatty acid-containing composition according to claim 17,
wherein the lower alcohol ester of fatty acid-containing composition has a ratio of an intensity of a peak which appears around 966 cm⁻¹ to an intensity of a peak which appears around 1,736 cm⁻¹ of 0.075 or less in FT-IR spectral analysis thereof.

21. The lower alcohol ester of fatty acid-containing composition according to claim 17,
wherein the lower alcohol ester of fatty acid-containing composition has an acid value of less than 5.

22-24. (canceled)

25. A lower alcohol ester of fatty acid-containing composition, comprising 96.5 mass % or more of a lower alcohol ester of EPA,
the lower alcohol ester of fatty acid-containing composition having a ratio of an intensity of a peak which appears around 966 cm⁻¹ to an intensity of a peak which appears around 1,736 cm⁻¹ of 0.085 or less in FT-IR spectral analysis thereof.

26-27. (canceled)
